(19) United States
(12) Patent Application Publication
(10) Pub. No.: US 2019/0142722 A1
(43) Pub. Date: May 16, 2019

(54) METHODS AND COMPOSITIONS FOR PROMOTING OR INDUCING HAIR GROWTH

(71) Applicant: THE TRUSTEES OF COLUMBIA UNIVERSITY IN THE CITY OF NEW YORK, NEW YORK, NY (US)

(72) Inventor: Angela M. Christiano, Mahwah, NJ (US)

(21) Appl. No.: 16/186,186

(22) Filed: Nov. 9, 2018

Related U.S. Application Data
(60) Provisional application No. 62/584,438, filed on Nov. 10, 2017.

(51) Int. Cl. A61K 8/64; A61Q 7/00; A61K 8/60

(52) U.S. Cl. CPC A61K 8/64; A61K 8/606; A61Q 7/00

(57) ABSTRACT

The presently disclosed subject matter relates, in certain embodiments, to compositions and methods for the inhibition of the Janus activated kinase-signal transducer and activator of transcription (JAK-STAT) pathway, and particularly inhibition of oncostatin (e.g. oncostatin M (OSM)), colony stimulating factor 1 receptor (CSF1R), interleukin-34 (IL-34), and/or trichophages, in order to induce or promote hair growth. In certain embodiments, the presently disclosed subject matter relates to topical treatments with small molecule inhibitors of the JAK-STAT pathway, and particularly small molecule inhibitors of oncostatin, CSF1R, IL-34, and/or trichophages, to induce or promote hair growth. Some embodiments are directed to treating hair loss disorders using inhibitors of oncostatin, CSF1R, IL-34 and/or trichophages. Embodiments also describe kits including inhibitors of oncostatin, CSF1R, IL-34 and/or trichophages.

### CROSS-REFERENCE TO RELATED APPLICATION

This application claims priority under 35 U.S.C. § 119(e) to U.S. Provisional Application Ser. No. 62/584,438, filed Nov. 10, 2017, the contents of which is incorporated herein in its entirety.

### STATEMENT OF GOVERNMENT INTEREST

This invention was made with government support under Grant No. AR070588 awarded by the National Institutes of Health. The government has certain rights in the invention.

### TECHNICAL FIELD

The presently disclosed subject matter relates, in certain embodiments, to compositions and methods for the inhibition of the Janus activated kinase-signal transducer and activator of transcription (JAK-STAT) pathway, and particularly inhibition of oncostatin (e.g. oncostatin M (OSM)), colony stimulating factor 1 receptor (CSF1R), interleukin 34 (IL-34), and/or trichophages, in order to induce or promote hair growth. In certain embodiments, the presently disclosed subject matter relates to topical treatments with small molecule inhibitors of the JAK-STAT pathway, and particularly small molecule inhibitors of oncostatin, CSF1R, IL-34, and/or trichophages, to induce or promote hair growth.

### BACKGROUND

Several hair growth disorders are characterized by the inability to re-enter the growth phase of the hair cycle (anagen). This can be, for example, due to hair follicle (HF) miniaturization in the case of androgenetic alopecia, or due to immune dysfunction in the case of alopecia areata. There is a need for pharmacologic agents to promote or induce hair growth in such hair loss disorders.

### SUMMARY

In certain embodiments, the present disclosure is directed to a method of inducing hair growth in a mammalian subject by administering to the subject a therapeutically effective amount of a CSF1R inhibitor. In certain embodiments, the present disclosure is directed to a method of inducing hair growth in a mammalian subject by administering to the subject a therapeutically effective amount of an IL-34 inhibitor. In certain embodiments, the present disclosure is directed to a method of inducing hair growth in a mammalian subject by administering to the subject a therapeutically effective amount of a trichophage inhibitor. In certain embodiments, the present disclosure is directed to a method of inducing hair growth in a mammalian subject by administering to the subject a therapeutically effective amount of an oncostatin inhibitor.

In certain other embodiments, the present disclosure is directed to a method of promoting hair growth in a mammalian subject by administering to the subject a therapeutically effective amount of a CSF1R inhibitor. In certain other embodiments, the present disclosure is directed to a method of promoting hair growth in a mammalian subject by administering to the subject a therapeutically effective amount of an IL-34 inhibitor. In certain other embodiments, the present disclosure is directed to a method of promoting hair growth in a mammalian subject by administering to the subject a therapeutically effective amount of a trichophage inhibitor. In some embodiments, the present disclosure is directed to the use of the oncostatin inhibitor, CSF1R inhibitor, IL-34 inhibitor, and/or trichophage inhibitor of embodiments herein to promote hair growth, induce hair growth, maintain the rate of hair growth, increase the rate of hair growth, decrease the rate of hair loss, prevent the onset or progression of a hair loss disorder, maintain remission in a subject having a hair loss disorder, improve remission in a subject having a hair loss disorder, prevent hair loss, improve the quality of the hair (e.g., increase density of hair, increase hair shaft strength or thickness), or the like. Such methods may be combined with one another and with any combinations of the following additional features:
i) administration is to a hair follicle, a part thereof, or a region thereof;
ii) administration occurs when the hair follicle is in telogen phase;
iii) the subject has non-scarring or scarring alopecia including, e.g., androgenetic alopecia (AGA), male and female pattern AGA, alopecia areata (AA), alopecia totalis (AT), alopecia universalis (AU), eyebrow alopecia, eyelash alopecia, intranasal hair alopecia, ophiasis pattern alopecia areata, sisaihpo pattern alopecia areata, male pattern hair loss, female pattern hair loss, anagen effluvium, telogen effluvium, hypotrichosis, hereditary hypotrichosis simplex, frontal fibrosing alopecia, cicatricial alopecia, lichen planopilaris, folliculitis decalvans, tufted folliculitis, dissecting cellulitis of the scalp, ring alopecia, chemotherapy induced alopecia, or superficial or deep infections of the scalp, e.g., tinea capitis.
iv) the inhibitor is an antisense RNA, an siRNA, an shRNA, a microRNA, or a variant or modification thereof that specifically inhibits expression of the gene that encodes CSF1R; or a small molecule;
v) the inhibitor may be selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof;
vi) the inhibitor is a CSF1R antibody, a CSF1 antibody, or an IL-34 antibody;
vii) the antibody may be selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), or a combination thereof.
viii) the subject is a human;
ix) the hair is on a scalp or a face, or constitutes an eyebrow or an eyelash of the subject, or any location on the body of the subject;
x) the hair is nasal hair;
xi) the inhibitor is administered topically;
xii) the inhibitor is administered orally;
xiii) the inhibitor is administered by injection;
xiv) an expression level of one or more hair growth biomarkers, CSF1R, and/or one or more trichophage biomarkers are changed after administering said inhibitor;
xv) the one or more hair growth biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17;
xvi) the gene expression change of one or more biomarkers are detected by quantitative PCR or a variation thereof;
xvii) the gene expression change of one or more biomarkers are detected by enzyme linked immunosorbant assay or a variation thereof;
xviii) the inhibitor may be any other inhibitor described herein in any formulation described herein.

In certain embodiments, the present disclosure also provides a kit for inducing or promoting hair growth in a mammalian subject. The kit includes an oncostatin inhibitor, a CSF1R inhibitor, trichophage inhibitor and/or an IL-34 inhibitor, and a pharmaceutically acceptable carrier. The inhibitor may be selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof. In some embodiments, the inhibitor may be an antibody selected from the group consisting of a CSF1R antibody, a CSF1 antibody, an IL-34 antibody, AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), SNDX-6352 (an IgG4 humanized monoclonal antibody that binds to the ligand binding domain of the CSF-1 receptor, blocking the binding and consequent activation by both natural ligands (IL-34 and CSF-1)), or a combination thereof. The kit can be used to implement any of the above methods.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1A is a photograph of mice treated in an assay.

FIG. 1B is a photograph of mice treated in an assay.

FIG. 1C is a Western blot.

FIG. 1D is a Western blot.

FIG. 1E is a photograph of the results of a colony forming assay (upper portion) and a graph of the results of the colony forming assay (lower portion).

FIG. 1F is a photograph of mice treated in an assay.

FIG. 2A is a set of graphs of relative expression of OSM and OSM receptor (OSMR) for various samples in an assay.

FIG. 2B is a set of photomicrographs of an immunofluorescence assay of dermal cells.

FIG. 2C is a Western blot (lower portion) and a graphical quantification of Western blot intensity (upper portion).

FIG. 2D is a set of photomicrographs of an immunofluorescence assay of dermal cells.

FIG. 2E is a photograph of mice treated.

FIG. 2F is a graph of the anagen induction index for various samples in an assay.

FIG. 2G is a photograph of mice treated in any assay.

FIG. 2H is a graph of the anagen induction index for various samples in an assay.

FIG. 2I is a set of graphs of relative expression of Krt17, OSMR, and IL6ST for various samples in an assay.

FIG. 2J is a graph of relative expression of OSMR for various samples in an assay.

FIG. 2K is a Western blot.

FIG. 2L is a set of photomicrographs of an immunofluorescence assay of dermal cells.

FIG. 2M is a set of graphs of relative expression of STAT5a and STAT5b for various samples in an assay.

FIG. 2N is a set of photomicrographs of an immunofluorescence assay of dermal cells.

FIG. 2O is a set of photomicrographs of an H&E immunohistochemistry assay of skin.

FIG. 2P is a set of photomicrographs of an immunofluorescence assay of dermal cells (lower portion) and a related timeline (upper portion).

FIG. 2Q is a graph of percent of epidermal cells with the indicated expression pattern in various samples in an assay.

FIG. 2R is a graph of the relative increase in population of indicated cells in various samples in an assay.

FIG. 3A is a graph or relative expression of OSM in the dermis at different times in an assay.

FIG. 3B is a photomicrograph of stained perifollicular dermal tissue (upper portion) and related Western blots (lower portion) in an assay.

FIG. 3C is a set of photomicrographs of RNAscope multiplex in situ hybridization in telogen skin (upper portion) and quantifications thereof (lower portion).

FIG. 3D is a set of graphs of flow cytometry analysis (left and middle portion) and a graph of quantified OSM expression (right portion) derived from the flow cytometry analysis.

FIG. 3E is a set of graphs of flow cytometry analysis (left and middle portion) and a graph of quantified OSM expression (right portion) derived from the flow cytometry analysis.

FIG. 3F is a set of graphs of flow cytometry analysis.

FIG. 4A is a TSNE-plot of results from a single-cell RNA sequencing assay for OSM (first image) with detailed results for a distinct cluster (second image).

FIG. 4B is a set of TSNE-plots of results from single-cell RNA sequencing assays for various genes.

FIG. 4C is a heat map of the results of gene-set enrichment analysis.

FIG. 4D is a bar graph of genes in OSM-producing macrophages as determined by an assay.

FIG. 4E is set TSNE-plots of results from a single-cell RNA sequencing assay for various genes.

FIG. 5A is a TSNE-plot of results from a single-cell RNA sequencing assay for OSM.

FIG. 5B is a set of TSNE-plots of results from single-cell RNA sequencing assays for various genes.

FIG. 5C is a set of plots of the results of immunofluorescence studies for expression of various proteins.

FIG. 5D is a set of plots of the results of immunofluorescence studies for expression of various proteins.

FIG. 6A is a photograph of mice treated in an assay.

FIG. 6B is a photograph of mice treated in an assay.

FIG. 6C is a photograph of mice treated in an assay.

FIG. 6D is a photograph of a mouse treated in an assay (upper portion) and a set of photomicrographs of an immunofluorescence assay of dermal cells from the indicated areas of the mouse skin (lower portion).

FIG. 6E is a graph of the effects of ablation of HF-associated macrophages (on HFSC proliferation.

FIG. 6F is a set of graphs of flow cytometry analysis.

FIG. 6G is a set of photographs of the results of a patch assay (upper portion) and a graph quantifying these results (lower portion).

FIG. 6H is a graph of relative expression of OSM for various samples in an assay.

FIG. 6I is a set of photographs of the results of a patch assay (upper portion) and a graph quantifying these results (lower portion).

FIG. 7 is a set of photomicrographs of an immunofluorescence assay in balding or non-balding skin.

FIG. 8 is a schematic diagram of the effects of trichophages (Trem2+ tissue-resident macrophages) on hair grown during different phases.

### DETAILED DESCRIPTION

For clarity and not by way of limitation the detailed description of the invention is divided into the following subsections:
5.1 Definitions
5.2 Trichophages, CSF1R, OSM, IL-34 and the JAK-STAT Pathway
5.3 Methods of Treatment
5.4 Pharmaceutical Compositions and Administration
5.5 Methods of Monitoring Efficacy of Treatment
5.6 Kits

Recently, the inventors demonstrated that pharmacological inhibition of the JAK-STAT pathway promotes rapid hair regrowth in alopecia areata (AA) in both mice and humans (1) (WO2013149194 A1 to Christiano, et al., incorporated herein). Unexpectedly, during the course of the studies on mice with AA, it was observed that topical treatment with JAK-STAT inhibitors resulted in an unusually robust hair growth, suggesting a localized effect on initiation of the hair cycle.

As demonstrated in PCT/US16/031541 to Christiano, et al., pharmacological inhibition of JAK-STAT signaling initiates the hair cycle in normal mice and promotes hair growth in humans. Particularly, in normal telogen phase, JAK signaling is elevated and is involved in maintaining quiescence of hair follicle stem cells. Administering a JAK inhibitor results in entry into anagen accompanied by hair growth by lowering the threshold of JAK signaling so that the hair follicle is no longer quiescent.

In connection with the present disclosure, the inventors have identified upstream factors that signal via the JAK-STAT pathway to promote hair growth. Oncostatin M (OSM) is one such upstream factor. OSM receptor (OSMR) is expressed on hair follicle stem cells, and all JAKs (JAK1, JAK2, and JAK3) as well as tyrosine kinase 2 (Tyk 2) are activated by OSM. Signaling from the JAKs and Tyk2 is transduced by STATS, primarily via STAT5, in hair follicle keratinocytes.

OSM was expected to be produced in a different compartment of the hair follicle from where the stem cells were located, such as in the dermal papilla. However, extensive experiments to identify OSM in the hair follicle were negative. Unexpectedly, “M2-like” anti-inflammatory macrophages, that are resident in the tissues near hair follicles, were identified as the source of OSM that promotes hair growth in the follicle. The macrophages identified herein as associated with OSM production are positive for triggering receptor expressed on myeloid cells 2 (TREM2), but have distinct differences from the typical M2 type and more closely resemble microglia. These TREM2+, OSM+, “M2-like” macrophages are referred to as “trichophages”.

Trichophages also exhibit a macrophage-specific marker, CSF1R. This marker was used to establish that targeting the trichophages or blocking CSF1R leads to hair growth due to the reduction or elimination of the source of OSM that acts on the hair follicle. CSF1R was also demonstrated to be a suitable target for topically applied small molecule inhibitors, which also led to hair regrowth.

Additionally, a second ligand for colony-stimulating factor-1 receptor (CSF-1R) with distinct biologic activities, interleukin 34 (IL-34), has recently been characterized. IL-34 and CSF1 signal through the common receptor (CSF1R) to mediate the biology of mononuclear phagocytic cells and aberrant macrophage activation by CSF1 and/or IL-34 is associated with numerous diseases. Although IL-34 and CSF-1 have some distinct activities under physiologic conditions, they appear functionally redundant in various disease states. Thus, blocking the activity of one (either) or both may be therapeutically efficacious.

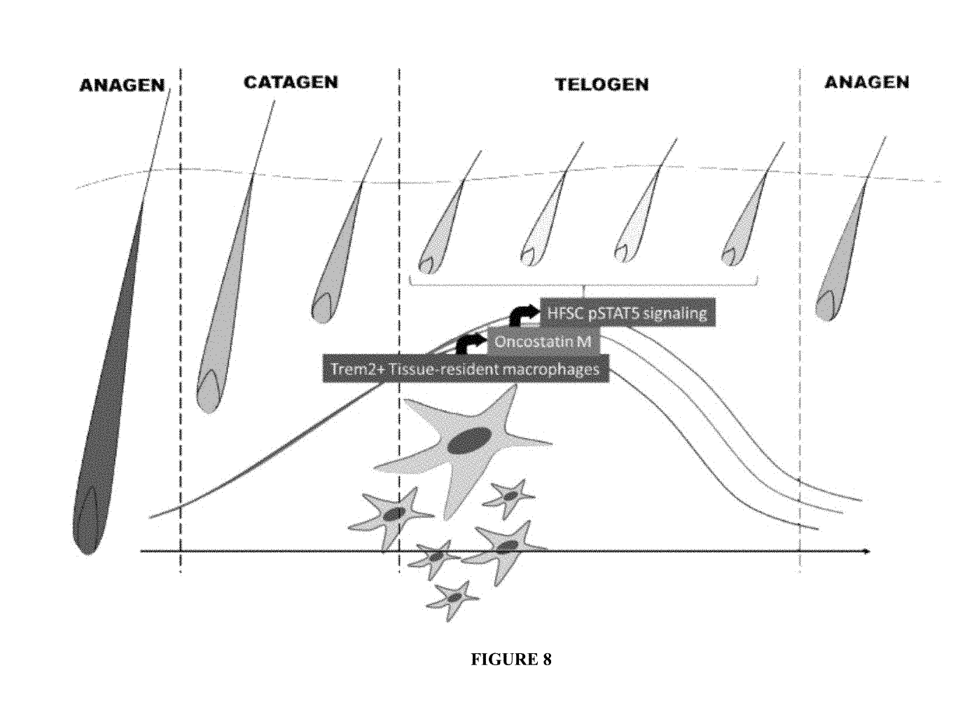

As illustrated in FIG. 8, trichophages produce OSM during early- and mid-telogen, which signals via JAK-STAT5 in hair follicle stem cells (HFSC) to inhibit proliferation and maintain their quiescence, particularly during second telogen in the mouse. These trichophages predominate during early and mid-telogen, and lose their OSM-producing ability as telogen progresses, which allows the hair follicles (HFs) to enter the next anagen stage.

The B6 anagen growth model has been widely used as a predictive model of hair growth in humans. It is believed that such trichophages also predominate during the early and mid-telogen stages in humans and are a major factor in hair loss and prevention of hair growth in hair loss disorders. Accordingly, the mouse models disclosed herein are highly predictive of responses in hair loss disorders of humans.

In light of the foregoing, in certain embodiments, the presently disclosed subject matter relates to compositions and methods for the inhibition of the JAK-STAT pathway, and particularly inhibition of oncostatin, CSF1R, IL-34, and/or trichophages, in order to induce hair growth. In certain embodiments, the presently disclosed subject matter relates to topical treatments with small molecule inhibitors of the JAK-STAT pathway, and particularly small molecule inhibitors of CSF1R and/or trichophages, to induce hair growth.

### Definitions

As used herein, the term “about” means plus or minus 10% of the numerical value of the number with which it is being used. Therefore, about 50% means in the range of 45%-55%.

According to the present disclosure, a “subject” or a “patient” is a human or non-human animal. Although the animal subject is preferably a human, the compounds and compositions of the invention have application in veterinary medicine as well, e.g., for the treatment of domesticated species such as canine, feline, murine, and various other pets; farm animal species such as bovine, equine, ovine, caprine, porcine, etc.; and wild animals, e.g., in the wild or in a zoological garden, such as non-human primates.

The terms “treat,” “treated,” or “treating” as used herein refers to both therapeutic treatment and prophylactic or preventative measures, wherein the object is to inhibit, prevent or slow down (lessen) an undesired physiological condition, disorder or disease, or to improve, inhibit, or otherwise obtain beneficial or desired clinical results. For the purposes of this disclosure, beneficial or desired clinical results include, but are not limited to, improvement or alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. For example, as used herein, treatment of a hair loss disorder could include promoting hair growth, inducing hair growth, maintaining the rate of hair growth, increasing the rate of hair growth, decreasing the rate of hair loss, preventing the onset or progression of a hair loss disorder, maintaining remission in a subject having a hair loss disorder, improving remission in a subject having a hair loss disorder, preventing hair loss, or the like.

“Pharmaceutical composition” and “pharmaceutical formulation,” as used herein, refer to a composition which is in such form as to permit the biological activity of an active ingredient contained therein to be effective, and which contains no additional components which are unacceptably toxic to a patient to which the formulation would be administered.

“Pharmaceutically acceptable,” as used herein, e.g., with respect to a “pharmaceutically acceptable carrier,” refers to the property of being nontoxic to a subject. A pharmaceutically acceptable ingredient in a pharmaceutical formulation can be an ingredient other than an active ingredient which is nontoxic. A pharmaceutically acceptable carrier can include, without limitation, a buffer, excipient, stabilizer, and/or preservative.

A “trichophage” as used herein refers to a tissue resident TREM2+ macrophage with an anti-inflammatory phenotype (e.g., CD163+ and/or CD206+) that produces OSM.

As used herein, a “CSF1R inhibitor” refers to a compound that interacts with either the receptor (CSF1R) or a ligand, e.g. CSF1, Interleukin-34 (IL-34), a CSF1R gene or a CSF1R protein or polypeptide and inhibits its activity and/or expression and/or targets a cell expressing a CSF1R protein or polypeptide.

As used herein, “hair follicle” refers to the sheath of cells and connective tissue that surrounds the root of a hair, including, e.g., the papilla, the germinal matrix, the hair bulb and the hair bulge (follicular stem-cell compartment). In some embodiments, the hair follicle could be in growth (anagen) phase. In some embodiments, the hair follicles may be in cessation (catagen) phase. In some embodiments, the hair follicle may be in rest (telogen) phase. In some embodiments, the hair follicles administered to may be in more than one phase. Hair follicles may be in various phases in a person. The time these phases last also varies from person to person. Different hair color and follicle shape may also affect the timings of these phases.

As used herein a “trichophage inhibitor” refers to a compound that interacts with a trichophage to kill the trichophage and/or to decrease or halt its production of OSM.

An inhibitor of the present disclosure can be a protein, such as an antibody (monoclonal, polyclonal, humanized, chimeric, or fully human), or a binding fragment thereof, directed against a polypeptide encoded by the corresponding sequence disclosed herein. An antibody fragment can be a form of an antibody other than the full-length form and includes portions or components that exist within full-length antibodies, in addition to antibody fragments that have been engineered. Antibody fragments can include, but are not limited to, single chain Fv (scFv), diabodies, Fv, and (Fab′)₂, triabodies, Fc, Fab, CDR1, CDR2, CDR3, combinations of CDR's, variable regions, tetrabodies, bifunctional hybrid antibodies, framework regions, constant regions, and the like (see, Maynard et al., (2000) Ann. Rev. Biomed. Eng. 2:339-76; Hudson (1998) Curr. Opin. Biotechnol. 9:395-402). Antibodies can be obtained commercially, custom generated, or synthesized against an antigen of interest according to methods established in the art (Janeway et al., (2001) Immunobiology, 5th ed., Garland Publishing). In some embodiments, the agent or inhibitor is a large molecule, protein, or antibody or a binding fragment thereof that binds, interacts, or associates with a target protein or ligand (e.g., CSF1, IL-34).

An inhibitor of the present disclosure can be a small molecule that binds to a protein and disrupts its function. Small molecules are a diverse group of synthetic and natural substances generally having low molecular weights. They can be isolated from natural sources (for example, plants, fungi, microbes and the like), are obtained commercially and/or available as libraries or collections, or synthesized. Candidate small molecules that modulate a protein can be identified via in silico screening or high-through-put (HTP) screening of combinatorial libraries. Most conventional pharmaceuticals, such as aspirin, penicillin, and many chemotherapeutics, are small molecules, can be obtained commercially, can be chemically synthesized, or can be obtained from random or combinatorial libraries (Werner et al., (2006) BriefFunct. Genomic Proteomic 5(1):32-6). In some embodiments, the agent is a small molecule that binds, interacts, or associates with a target protein or RNA, e.g. CSF1R. Such a small molecule can be an organic molecule that, when the target is an intracellular target, is capable of penetrating the lipid bilayer of a cell to interact with the target. Small molecules include, but are not limited to, toxins, chelating agents, metals, and metalloid compounds. Small molecules can be attached or conjugated to a targeting agent so as to specifically guide the small molecule to a particular cell.

As used herein, “therapeutically effective amount” refers to the amount of the inhibitors of the present disclosure contained in the composition administered is of sufficient quantity to achieve the intended purpose, such as, in this case, to induce or promote hair growth, to prevent hair loss, or to reduce hair loss in the subject. For the purpose of the present disclosure, methods of measuring hair growth are well known in the art and need not be repeated herein. In the context of administering an inhibitor to induce hair growth, an effective amount of a composition is an amount sufficient to cause the hair follicle to re-enter anagen phase, to prolong the anagen phase, to decrease the telogen phase, or to otherwise result in the effect of increasing the quantity or quality of hair growth (e.g., increased thickness of the hair shaft, increased density of hair, and the like). In the context of administering an inhibitor to promote hair growth, an effective amount of an inhibitor is an amount sufficient to increase the rate of hair growth, to decrease the rate of hair loss, or to otherwise result in the effect of increasing the quantity or quality of hair growth (e.g., increased thickness of the hair shaft, increased velocity of hair growth, increased density of hair, and the like). For example, the increase can be a 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 100%, 200%, 300%, 400%, 500%, 600%, 700%, 800%, 900%, 1000%, 5000%, 10000% or more increase in the rate of hair growth. A therapeutically effective amount for each administration can be any amount between 1 ng to 1 ug, 1 ug to 1 mg, 2 mg, 3 mg, 4 mg, 5 mg, 6 mg, 7 mg, 8 mg, 9 mg, 10 mg, 20 mg, 30 mg, 40 mg, 50 mg, 100 mg, 200 mg, 300 mg, 400 mg, 500 mg, 1 g or more, or any intermediate amount thereof. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, concomitant therapies and the condition being treated. The compounds are effective over a wide dosage range and, for example, dosages per day will normally fall within the range of from about 0.0001 μg/kg to about 40,000 μg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of embodiments herein in any way. A therapeutically effective amount can be administered in one or more administrations. A therapeutically effective amount of the inhibitors can be administered topically, orally or intravenously. When used in an admixture with a pharmaceutically acceptable diluent, carrier or excipient, an effective amount of an inhibitor can be an amount of 0.01%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or more than 10% by weight, or any intermediate amount thereof.

As used herein, “anagen” or “anagen phase” refers to the active growth phase of hair follicles. Typically, in anagen phase, the cells at the base of the hair follicle are dividing rapidly, generating the hair shaft. At the end of the anagen phase, certain biological signals cause the follicle to enter the catagen phase.

As used herein, “catagen” or “catagen phase” refers to a transition stage that occurs at the end of the anagen phase. It signals the end of the active growth of a hair.

As used herein, “telogen” or “telogen phase” refers to the resting phase of the hair follicle. During the telogen phase the follicle remains dormant. In response to certain biological signals, the follicle will re-enter anagen phase and the hair shaft will begin to grow again.

As used herein, “androgenetic alopecia”, also known as male-pattern or female-pattern hair loss, is hair loss that occurs due to an underlying susceptibility of hair follicles to miniaturize due in part to the influence of androgenic hormones and/or other immunomodulatory factors.

As used herein, “telogen effluvium” is a scalp disorder characterized by the thinning or shedding of hair resulting from the coordinated entry of hair in the telogen phase (the resting phase of the hair follicle)

As used herein, “alopecia areata” is an autoimmune disease in which hair is lost from some or all areas of the body due to the body's aberrant recognition of its own cells as foreign and subsequent destruction of its own tissue.

As used herein, “patchy alopecia” refers to a form of alopecia areata characterized by the loss of one or more patches of hair from anywhere on the body including, e.g., scalp hair, facial hair including eyelashes, eyebrows and/or nasal hair.

As used herein, “alopecia totalis” refers to a form of alopecia areata characterized by the loss of all hair on the scalp (and may include eyebrow hair loss).

As used herein, “alopecia universalis” refers to a condition characterized by the complete loss of hair on the scalp and body including loss of eyelashes, eyebrows and nasal hair. It is an advanced form of alopecia areata.

As used herein, “tinea capitis”, is a cutaneous fungal infection (dermatophytosis) of the scalp. The disease is primarily caused by dermatophytes in the Trichophyton and Microsporum genera that invade the hair shaft. The clinical presentation is typically single or multiple patches of hair loss, sometimes with a ‘black dot’ pattern (often with broken-off hairs), that may be accompanied by inflammation, scaling, pustules, and itching.

As used herein, “hypotrichosis” refers to a condition of abnormal hair patterns—predominantly loss or reduction.

As used herein, “hereditary hypotrichosis simplex” refers to a genetic disorder, characterized by sparse or absent scalp hair, in the absence of other ectodermal or systemic abnormalities.

As used herein, “frontal fibrosing alopecia” refers to a form of scarring hair loss affecting the hair margin on the front of the scalp.

As used herein, “cicatricial alopecia” also called scarring alopecia, refers to a group of inflammatory disorders that destroy hair follicles. The follicles are replaced with scar tissue, causing permanent hair loss.

As used herein, “lichen planopilaris” is a type of scarring hair loss that occurs when a relatively common skin disease, known as lichen planus, affects areas of the skin with hair. Lichen planopilaris destroys the hair follicle replacing it with scarring.

As used herein, “ring alopecia” is a ring or band of alopecia encircling or partially encircling the head; it may extend along the posterior occipital area, around the temporal portion of the scalp above the ears or onto the forehead.

As used herein, “chemotherapy induced alopecia” is a type of hair loss that occurs after chemotherapy for treatment of cancer or non-cancer diseases such as lupus and rheumatoid arthritis.

### Trichophages, CSF1R, Oncostatin, IL-34 and the JAK-STAT Pathway

The JAK-STAT signalling pathway transmits biological signals from extracellular environment to the nucleus and causes DNA transcription and expression of genes involved in differentiation, apoptosis, immunity, proliferation, and oncogenesis. The three main components of the pathway are a cell surface receptor, a JAK protein, and a STAT protein.

JAKs are a family of intracellular, nonreceptor tyrosine kinases. STATs are a family of transcription factors. The binding of ligands such as interferon, interleukin, and/or growth factors to cell surface receptors activate associated JAKs, which phosphorylate tyrosine residues on the receptor, creating binding sites for SH2 domains. STATs, which contain SH2 domains, are then recruited to the receptor whereby they are also tyrosine-phosphorylated by JAKs. The activated STATs form heterodimers or homodimers and translocate to the cell nucleus to induce transcription of target genes. STATs may also be tyrosine-phosphorylated directly by receptor tyrosine kinases (e.g., epidermal growth factor receptor) and/or non-receptor tyrosine kinases (e.g., c-src).

The JAK family of genes comprises Janus kinase 1 (JAK1, GenBank ID: 3716), Janus kinase 2 (JAK2, GenBank ID: 3717), Janus kinase 3 (JAK3, GenBank ID: 3718), and Tyrosine kinase 2 (TYK2, GenBank ID: 7297).

The STAT family genes comprises signal transducer and activator of transcription 1 (STAT1, GenBank ID: 6772), signal transducer and activator of transcription 2 (STAT2, GenBank ID: 6773), signal transducer and activator of transcription 3 (STAT3, GenBank ID: 6774), signal transducer and activator of transcription 4 (STAT4, GenBank ID: 6775), signal transducer and activator of transcription 5A (STAT5A, GenBank ID: 6776), signal transducer and activator of transcription 5B (STAT5B, GenBank ID: 6777), and signal transducer and activator of transcription 6 (STAT6, GenBank ID: 6778).

Oncostatin M (OSM, GenBank ID: 5008) is a gene encoding a member of the leukemia inhibitory factor/oncostatin-M (LIF/OSM) family of proteins. The encoded preproprotein is proteolytically processed to generate the mature protein. This protein is a secreted cytokine and growth regulator that inhibits the proliferation of a number of tumor cell lines. This protein also regulates the production of other cytokines, including interleukin 6, granulocyte-colony stimulating factor and granulocyte-macrophage colony stimulating factor in endothelial cells. OSM mediates its bioactivities through two different heterodimer receptors. The gp130 receptor is the common component, which dimerizes with either leukemia inhibitory factor receptor (LIFR) or with OSM receptor β (OSM-Rβ) to generate, respectively, type I and type II OSM receptors. Both type I and type II OSM receptors activate the JAK-STAT signal pathway.

In the mouse, administration of recombinant OSM was shown previously to be a potent inhibitor of anagen (Yu, M., et al., Interleukin-6 cytokine family member oncostatin M is a hair-follicle-expressed factor with hair growth inhibitory properties. Exp Dermatol, 2008. 17(1): p. 12-9.), but until now, its source and physiological contribution to the hair cycle remained undefined. In humans, OSM was first discovered as a negative growth regulator of the A375 melanoma cell line (Zarling, J. M., et al., Oncostatin M: a growth regulator produced by differentiated histiocytic lymphoma cells. Proc Natl Acad Sci USA, 1986. 83(24): p. 9739-43), at a time when researchers were searching for endogenous regulators of cancer cell growth. While OSM was first isolated in the supernatant of histiocytic lymphoma cells (Zarling, 1986), it was found to be more reliably obtained from macrophage cell lines (Malik, N., et al., Molecular cloning, sequence analysis, and functional expression of a novel growth regulator, oncostatin M. Mol Cell Biol, 1989. 9(7): p. 2847-53). OSM was found to have pleiotropic properties and its effects have been shown to be tissue- and context-dependent, acting as a growth inhibitor in some cell lines, and as an activator in others (Horn, D., et al., Regulation of cell growth by recombinant oncostatin M. Growth Factors, 1990. 2(2-3): p. 157-65; Dey, G., et al., Signaling network of Oncostatin M pathway. J Cell Commun Signal, 2013. 7(2): p. 103-8).

OSM has also been shown to play roles in development, inflammation and hematopoiesis (Gomez-Lechon, M. J., Oncostatin M: signal transduction and biological activity. Life Sci, 1999. 65(20): p. 2019-30; Hermanns, H. M., Oncostatin M and interleukin-31: Cytokines, receptors, signal transduction and physiology. Cytokine Growth Factor Rev, 2015. 26(5): p. 545-58). OSM appears to play a pro-inflammatory role in human keratinocytes (Boniface, K., et al., Oncostatin M secreted by skin infiltrating T lymphocytes is a potent keratinocyte activator involved in skin inflammation. J Immunol, 2007. 178(7): p. 4615-22; Pohin, M., et al., Oncostatin M overexpression induces skin inflammation but is not required in the mouse model of imiquimod-induced psoriasis-like inflammation. Eur J Immunol, 2016. 46(7): p. 1737-51), causing epidermal hyperplasia in the context of some diseases, but also having inhibitory effects on keratinocyte differentiation under other conditions (Rabeony, H., et al., Inhibition of keratinocyte differentiation by the synergistic effect of IL-17A, IL-22, IL-1alpha, TNFalpha and oncostatin M. PLoS One, 2014. 9(7): p. e101937). Lately, OSM is emerging as an important cytokine in tissue remodeling and regeneration in humans. OSM produced by M2-like macrophages during acute hepatic injury in mice was associated with a pro-fibrotic phenotype (Matsuda, M., et al., Oncostatin M causes liver fibrosis by regulating cooperation between hepatic stellate cells and macrophages in mice. Hepatology, 2017). Macrophage OSM has also been shown to induce bone formation by mesenchymal stem cells during physiological osteogenesis (Guihard, P., et al., Induction of osteogenesis in mesenchymal stem cells by activated onocytes/macrophages depends on oncostatin M signaling. Stem Cells, 2012. 30(4): p. 762-72), as well as in pathological Ewing sarcoma (David, E., et al., Oncostatin M is a growth factor for Ewing sarcoma. Am J Pathol, 2012. 181(5): p. 1782-95. OSM has also been shown to be secreted in excess by mononuclear cells of patients with systemic sclerosis (Hasegawa, M., et al., Enhanced production of interleukin-6 (IL-6), oncostatin M and soluble IL-6 receptor by cultured peripheral blood mononuclear cells from patients with systemic sclerosis. Rheumatology (Oxford), 1999. 38(7): p. 612-7), and has pro-fibrotic effects in the skin and lung (Ho, Y. Y., et al., Cells from the skin of patients with systemic sclerosis secrete chitinase 3-like protein 1. BBA Clin, 2014. 1: p. 2-11; Atamas, S. P. and B. White, Cytokine regulation of pulmonary fibrosis in scleroderma. Cytokine Growth Factor Rev, 2003. 14(6): p. 537-50). Monoclonal antibodies against OSM are currently undergoing Phase I and II trials for the treatment of systemic sclerosis (Clinical Trial # NCT03041025).

Glycoprotein 130 (gp130, GenBank ID: 3572, also known as interleukin 6 signal transducer, IL6ST, IL6-beta or CD130) is a transmembrane signal transducer protein shared by many cytokines, including interleukin 6 (IL6), ciliary neurotrophic factor (CNTF), leukemia inhibitory factor (LIF), and oncostatin M (OSM). This protein functions as a part of the cytokine receptor complex. The activation of this protein is dependent upon the binding of cytokines to their receptors.

OSM receptor β (OSM-Rβ, GenBank ID: 9180, also known as the oncostatin M receptor, or OSMR) is a gene encoding a member of the type I cytokine receptor family. The encoded protein heterodimerizes with gp130 to form the type II oncostatin M receptor and with interleukin 31 receptor A to form the interleukin 31 receptor, and thus transduces oncostatin M and interleukin 31 induced signaling events.

Leukemia inhibitory factor receptor (LIFR, GenBank ID: 3977, also known as leukemia inhibitory factor receptor alpha) is a gene encoding a protein that belongs to the type I cytokine receptor family. This protein combines with a high-affinity converter subunit, gp130, to form a receptor complex that mediates the action of the leukemia inhibitory factor, a polyfunctional cytokine that is involved in cellular differentiation, proliferation and survival in the adult and the embryo.

Macrophages have been described to cluster around hair follicles (Eichmuller, S., et al., Clusters of perifollicular macrophages in normal murine skin: physiological degeneration of selected hair follicles by programmed organ deletion. J Histochem Cytochem, 1998. 46(3): p. 361-70), and may secrete FGF-5 that promotes catagen (Suzuki, S., et al., Localization of rat FGF-5 protein in skin macrophage-like cells and FGF-5S protein in hair follicle: possible involvement of two Fgf-5 gene products in hair growth cycle regulation. J Invest Dermatol, 1998. 111(6): p. 963-72; Suzuki, S., et al., Dual-mode regulation of hair growth cycle by two Fgf-5 gene products. J Invest Dermatol, 2000. 114(3): p. 456-63).

Trichophages appear to be genetically similar to microglia and might perform similar functions with respect to the hair cycle. Microglia are important supportive cells of the brain and CNS, where they carry out innate immune functions, clear cell debris, and participate in homeostasis and pruning of neurons (Hanisch, U.K. and H. Kettenmann, Microglia: active sensor and versatile effector cells in the normal and pathologic brain. Nat Neurosci, 2007. 10(11): p. 1387-94). TREM2-DAP12 signaling in microglia have been functionally linked to their survival (Poliani, P. L., et al., TREM2 sustains microglial expansion during aging and response to demyelination. J Clin Invest, 2015. 125(5): p. 2161-70) and role in phagocytosis (Takahashi, K., et al., TREM2-transduced myeloid precursors mediate nervous tissue debris clearance and facilitate recovery in an animal model of multiple sclerosis. PLoS Med, 2007. 4(4): p. e124). This may be analogous to the role of trichophages in the hair cycle, where they modulate HFSC activity and coordinate the cyclical growth and regression of hair follicles. In the CNS, dysfunctional microglia have been implicated in the pathogenesis of Alzheimer's disease, and mutations in TREM2 have been associated with neurodegenerative disease in humans (Lue, L. F., C. Schmitz, and D. G. Walker, What happens to microglial TREM2 in Alzheimer's disease: Immunoregulatory turned into immunopathogenic?Neuroscience, 2015. 302: p. 138-50; Painter, M. M., et al., TREM2 in CNS homeostasis and neurodegenerative disease. Mol Neurodegener, 2015. 10: p. 43). Whole exome sequencing has implicated TREM2 in diseases such as polycystic lipomembranous osteodysplsia (PLOSL) (Dardiotis, E., et al., A novel mutation in TREM2 gene causing Nasu-Hakola disease and review of the literature. Neurobiol Aging, 2017. 53: p. 194 e13-194 e22) and frontotemporal dementia (FTD) (Guerreiro, R. J., et al., Using exome sequencing to reveal mutations in TREM2 presenting as a frontotemporal dementia-like syndrome without bone involvement. JAMA Neurol, 2013. 70(1): p. 78-84), but a distinct hair pathology has not been described in these patients. Dysfunctional trichophages may play a similar role in hair disorders such as AGA.

Macrophages are involved in other anagen-inducing processes as indicated in Table 1.

TABLE 1
Role of JAK-STAT signaling and immune cell involvement during the hair cycle

| Phase of hair | | Evidence for immune cell |
|---|---|---|
| cycle | Evidence for JAK-STAT signaling | involvement |
| Telogen (rest) | Pharmacological JAK-STAT | Macrophages predominate during |
| | inhibition induces anagen (Harel, | telogen, and clodronate |
| | S., et al., Pharmacologic inhibition | liposomes induce anagen |
| | of JAK-STAT signaling promotes | (Castellana, D., R. Paus, and M. |
| | hair growth. Sci Adv, 2015. 1(9): p. | Perez-Moreno, Macrophages |
| | e1500973) | contribute to the cyclic activation |
| | Prolactin-JAK-STAT5 signaling | of adult hair follicle stem cells. |
| | maintains HFSC quiescence during | PLoS Biol, 2014. 12(12): p. |
| | pregnancy and lactation (Goldstein, | e1002002). |
| | J., et al., Calcineurin/Nfatc1 | |
| | signaling links skin stem cell | |
| | quiescence to hormonal signaling | |
| | during pregnancy and lactation. | |
| | Genes Dev, 2014. 28(9): p. 983-94) | |
| Anagen | Constitutive epidermal STAT3 | At the end of telogen, macrophages |
| (spontaneous) | ablation inhibits first spontaneous | undergo apoptosis and release |
| | anagen (Sano, S., et al., Two | Wnt7a, which stimulates anagen |
| | distinct signaling pathways in hair | (Castellana, 2014). |
| | cycle induction: Stat3-dependent | |
| | and -independent pathways. Proc | |
| | Natl Acad Sci USA, 2000. 97(25): | |
| | p. 13824-9), but not plucking | |
| | induced anagen. | |
| Anagen | | T regulatory cells mediate anagen |
| (depilation | | via Jagged1-Notch signaling (Ali, N., |
| induced) | | et al., Regulatory T Cells in Skin |
| | | Facilitate Epithelial Stem Cell |
| | | Differentiation. Cell, 2017. 169(6): p. |
| | | 1119-1129 e11). |
| Anagen | | Inflammatory “M1-like” |
| (plucking | | macrophages are recruited to |
| induced) | | plucked HFs by CCL2, and produce |
| | | TNF-α that stimulates anagen |
| | | (Chen, C. C., et al., Organ-level |
| | | quorum sensing directs regeneration |
| | | in hair stem cell populations. Cell, |
| | | 2015. 161(2): p. 277-90). |
| Wound- | | γδ T cells produce FGF-9 that |
| induced HF | | facilitates new HF formation in a |
| neogenesis | | regenerating wound (Gay, D., et al., |
| (WIHN) | | Fgf9 from dermal gammadelta T |
| | | cells induces hair follicle neogenesis |
| | | after wounding. Nat Med, 2013. |
| | | 19(7): p. 916-23). |
| Catagen | IL-6 induced catagen in anagen HFs | Macrophages are recruited to clear |
| (regression) | (Kwack, M. H., et al., | regressing follicles (Eichmuller, S., |
| | Dihydrotestosterone-inducible IL-6 | et al., Clusters of perifollicular |
| | inhibits elongation of human hair | macrophages in normal murine skin: |
| | shafts by suppressing matrix cell | physiological degeneration of |
| | proliferation and promotes regression | selected hair follicles by |
| | of hair follicles in mice. J Invest | programmed organ deletion. J |
| | Dermatol, 2012. 132(1): p. 43-9). | Histochem Cytochem, 1998. 46(3): |
| | | p. 361-70), and produce FGF-5 that |
| | | facilitates catagen (Suzuki, S., et al., |
| | | Localization of rat FGF-5 protein in |
| | | skin macrophage-like cells and |
| | | FGF-5S protein in hair follicle: |
| | | possible involvement of two Fgf-5 |
| | | gene products in hair growth cycle |
| | | regulation. J Invest Dermatol, 1998. |
| | | 111(6): p. 963-72; Suzuki, S., et al., |
| | | Dual-mode regulation of hair growth |
| | | cycle by two Fgf-5 gene products. J |
| | | Invest Dermatol, 2000. 114(3): p. |
| | | 456-63) . . . |

For example, in plucking induced anagen, damaged hair follicles release CCL2, which attract TNF-producing “M1-like” inflammatory macrophages that initiate anagen in surrounding follicles (Chen, C. C., et al., Organ-level quorum sensing directs regeneration in hair stem cell populations. Cell, 2015. 161(2): p. 277-90), a function that is opposite to the role of the trichophage. This pathway may have evolved in order to maintain a coat of fur in rodents following environmental or predator-inflicted damage. Further, the fluctuations of the immune system with the hair cycle have been described (Botchkarev, V. A., et al., Hair cycle-dependent changes in mast cell histochemistry in murine skin. Arch Dermatol Res, 1995. 287(7): p. 683-6; Hoffman, U., et al., Hair cycle-dependent changes in skin immune functions: anagen-associated depression of sensitization for contact hypersensitivity in mice. J Invest Dermatol, 1996. 106(4): p. 598-604), and parts of it have been elucidated. For example, depilation-induced anagen has been shown to be mediated by Jag1-Notch signaling from T regulatory cells (Ali, N., et al., Regulatory T Cells in Skin Facilitate Epithelial Stem Cell Differentiation. Cell, 2017. 169(6): p. 1119-1129 ell; Paus, R., et al., Distribution and changing density of gamma-delta T cells in murine skin during the induced hair cycle. Br J Dermatol, 1994. 130(3): p. 281-9), and wound-induced hair follicle neogenesis (WIHN) involves recruitment of FGF9-secreting γδ T cells (Gay, D., et al., Fgf9 from dermal gammadelta T cells induces hair follicle neogenesis after wounding. Nat Med, 2013. 19(7): p. 916-23). T regs do not appear to be involved in the spontaneous anagen initiated by JAK inhibition, or tricohophage inhibition/depletion, as they do in anagen resulting from depilation injury. It is possible that trichophages differentiate into “M1-like” macrophages in response to plucking and they may produce chemokines or factors that recruit other cell types that influence the hair cycle.

Murine HFSCs of the bulge and HG express both the receptor (OSMR) and co-receptor (gp130) necessary for OSM signaling, which occurs via the JAK-STAT and MAPK signaling pathways. STAT5 is the most likely downstream mediator of quiescence in HFSC because the activated phosphorylated form of STAT5 in the HFSC coincides with the early-to-mid second telogen, and its genetic ablation is sufficient for prematurely initiating anagen during telogen. In addition to its inhibitory properties on melanoma cell lines, OSM signaling via the JAK-STAT5 pathway inhibits adipocyte terminal differentiation (Miyaoka, Y., et al., Oncostatin M inhibits adipogenesis through the RAS/ERK and STAT5 signaling pathways. J Biol Chem, 2006. 281(49): p. 37913-20), delays cell cycle entry in HepG2 cells (Klausen, P., et al., Oncostatin M and interleukin 6 inhibit cell cycle progression by prevention of p27kip1 degradation in HepG2 cells. Oncogene, 2000. 19(32): p. 3675-83), and suppresses cytokine secretion in T cells (Hintzen, C., et al., Oncostatin M-induced and constitutive activation of the JAK2/STAT5/CIS pathway suppresses CCL1, but not CCL7 and CCL8, chemokine expression. J Immunol, 2008. 181(10): p. 7341-9). Further, the murine OSMRβ subunit is believed to directly recruit JAK2 to phosphorylate STAT5 in response to ligand binding (Hintzen, C., et al., Box 2 region of the oncostatin M receptor determines specificity for recruitment of Janus kinases and STAT5 activation. J Biol Chem, 2008. 283(28): p. 19465-77).

The maintenance of quiescence via JAK-STAT signaling is emerging as a common theme in different organisms and cell types, where its role is evolutionary conserved. JAK-STAT signaling in the Drosophila testis is mediated by the ligand Unpaired, which is the fly homologue of IL-6, and signals via STAT92E to prevent differentiation of the germline stem cells (Bausek, N., JAK-STAT signaling in stem cells and their niches in Drosophila. JAKSTAT, 2013. 2(3): p. e25686). JAK2-STAT5 signaling in murine hepatic stellate stem cells mediates quiescence signals from vitamin A and insulin (Yoneda, A., et al., Vitamin A and insulin are required for the maintenance of hepatic stellate cell quiescence. Exp Cell Res, 2016. 341(1): p. 8-17). In the murine mammary gland, another organ that undergoes controlled cycles of growth and involution, JAK-STAT3 transmits signals via LIF (another gp130-dependent cytokine) to mediate involution (Humphreys, R. C., et al., Deletion of Stat3 blocks mammary gland involution and extends functional competence of the secretory epithelium in the absence of lactogenic stimuli. Endocrinology, 2002. 143(9): p. 3641-50), while JAK-STAT5 transmits signals from Prolactin (PRL) during lactation to increase milk production (Hughes, K. and C. J. Watson, The spectrum of STAT functions in mammary gland development. JAKSTAT, 2012. 1(3): p. 151-8). Interestingly, during pregnancy and lactation, PRL signaling occurs via JAK-STAT5 in the HFSC to maintain quiescence of the hair follicles, perhaps to conserve nutritional resources during pregnancy (Goldstein, J., et al., Calcineurin/Nfatc1 signaling links skin stem cell quiescence to hormonal signaling during pregnancy and lactation. Genes Dev, 2014. 28(9): p. 983-94; Foitzik, K., et al., Prolactin and its receptor are expressed in murine hair follicle epithelium, show hair cycle-dependent expression, and induce catagen. Am J Pathol, 2003. 162(5): p. 1611-21). In this disclosure, it is demonstrated that the JAK-STAT5 pathway functions downstream of OSM to maintain HFSC quiescence is physiologically relevant during telogen.

JAK-STAT3 signaling has been shown to be required for the initiation of spontaneous anagen, but not for plucking-induced anagen, in mice (Sano, S., et al., Two distinct signaling pathways in hair cycle induction: Stat3-dependent and-independent pathways. Proc Natl Acad Sci USA, 2000. 97(25): p. 13824-9). Both STAT3 and STAT5 are dynamically expressed across telogen, but only pSTAT5 is specific for the HFSC during this phase. Further, using the covalent JAK3 inhibitor PF-06651600, it has been demonstrated that that inhibition of the JAK3-STAT5 signaling axis alone in HFSCs is sufficient to initiate anagen. The role of STAT3 in keratinocyte differentiation and migration is distinct from the role of STAT5 in maintaining HFSC quiescence. STAT3 and STAT5 signaling likely contribute to the opposing processes of quiescence and proliferation/migration, and may interact in the coordination of the induced hair cycle (e.g. in response to plucking, depilation and wounding).

JAK inhibitors that have been FDA approved for the treatment of rheumatoid arthritis (RA) (Tofacitinib), and myelofibrosis (Ruxolitinib) have been shown to be efficacious in the treatment of alopecia areata (AA), an autoimmune form of hair loss (Mackay-Wiggan, J., et al., Oral ruxolitinib induces hair regrowth in patients with moderate-to-severe alopecia areata. JCI Insight, 2016. 1(15): p. e89790; Kennedy Crispin, M., et al., Safety and efficacy of the JAK inhibitor tofacitinib citrate in patients with alopecia areata. JCI Insight, 2016. 1(15): p. e89776), where their mode of action likely lies in the inhibition of pathogenic NKG2D+ CD8+ T cells. Topical formulations of JAK inhibitors have induced a more robust anagen than systemic treatment (Harel, S., et al., Pharmacologic inhibition of JAK-STAT signaling promotes hair growth. Sci Adv, 2015. 1(9): p. e15009731; Xing, L., et al., Alopecia areata is driven by cytotoxic T lymphocytes and is reversed by JAK inhibition. Nat Med, 2014. 20(9): p. 1043-9).

### Methods of Treatment

In some embodiments, the present disclosure is directed to the use of the oncostatin inhibitor, CSF1R inhibitor, IL-34 inhibitor, and/or trichophage inhibitor of embodiments herein to promote hair growth, induce hair growth, maintain the rate of hair growth, increase the rate of hair growth, decrease the rate of hair loss, prevent the onset or progression of a hair loss disorder, maintain remission in a subject having a hair loss disorder, improve remission in a subject having a hair loss disorder, prevent hair loss, or the like. In some embodiments, the oncostatin inhibitor, CSF1R inhibitor, IL-34 inhibitor, and/or trichophage inhibitor of embodiments herein are administered in a therapeutically effective amount. In some embodiments, the oncostatin inhibitor, CSF1R inhibitor, IL-34 inhibitor, and/or trichophage inhibitor of embodiments herein are administered as a pharmaceutical composition further comprising a pharmaceutically acceptable excipient. Such inhibitors may also be used to treat hair loss disorders.

In some embodiments, the present disclosure relates to the use of a therapeutically effective amount of one or more CSF1R inhibitors to induce or promote hair growth. The present disclosure further relates to the use of a therapeutically effective amount of one or more oncostatin (e.g. OSM) inhibitors to induce or promote hair growth. The present disclosure further relates to the use of a therapeutically effective amount of one or more IL-34 inhibitors to induce or promote hair growth. The present disclosure further relates to the use of a therapeutically effective amount of one or more trichophage inhibitors to induce or promote hair growth.

In some embodiments, the antibody may be selected from antibodies targeting the ligands that signal through CSF1R. In some embodiments, the antibody may target CSF1, IL-34, or a combination thereof. In some embodiments the antibody may be a sequestering antibody for such ligands.

In some embodiments the antibody may be selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), SNDX-6352 (an IgG4 humanized monoclonal antibody that binds to the ligand binding domain of the CSF-1 receptor, blocking the binding and consequent activation by both natural ligands (IL-34 and CSF-1)), or a combination thereof.

In some embodiments, the small molecule inhibitor that targets the CSF1R receptor may be selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945)); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

A therapeutically effective amount of the one or more CSF1R inhibitors may be an amount sufficient to kill trichophages expressing CSF1R, or to decrease their OSM production sufficiently to induce or promote hair growth. Neutralizing antibodies that bind specifically to the same CSF1R domain may also be used. Small molecule derivatives of pexidartinib, which share a common chemical structure and formula, with substitutions that do not significantly decrease its ability to inhibit CSF1R, may also be used.

The present disclosure further relates to the use of a therapeutically effective amount of one or more trichophage inhibitors. Trichophage inhibitors may include CSF1R inhibitors or oncostatin inhibitors, and may also include other small molecules or proteins that kill trichophages and/or decrease their OSM production, such that hair growth is induced or promoted. Trichophage inhibitors may have effects specific to trichophages, of they may have more generalized effects. Trichophage inhibitors having more generalized effects may, in particular, be administered topically to the area where hair growth is induced or promoted to minimize side effects in other parts of the body.

Non-limiting contexts where such induction or promotion of hair growth would be desirable include, but are not limited to, those contexts where a subject has hair loss such as resulting from a non-scarring or scarring alopecia including, e.g., androgenetic alopecia (AGA), male and female pattern AGA, alopecia areata (AA), alopecia totalis (AT), alopecia universalis (AU), eyebrow alopecia, eyelash alopecia, intranasal hair alopecia, ophiasis pattern alopecia areata, sisaihpo pattern alopecia areata, male pattern hair loss, female pattern hair loss, anagen effluvium, telogen effluvium, tinea capitis, hypotrichosis, hereditary hypotrichosis simplex, frontal fibrosing alopecia, cicatricial alopecia, lichen planopilaris, folliculitis decalvans, tufted folliculitis, dissecting cellulitis of the scalp, ring alopecia or chemotherapy induced alopecia. In some embodiments, a method of treating a hair loss disorder in a subject in need thereof comprises administering to the subject a therapeutically effective amount of a oncostatin inhibitor, a CSF1R inhibitor, an IL-34 inhibitor, and/or a trichophage inhibitor. In some embodiments, the hair loss disorder is selected from non-scarring or scarring alopecia including, e.g., androgenetic alopecia (AGA), male and female pattern AGA, alopecia areata (AA), alopecia totalis (AT), alopecia universalis (AU), eyebrow alopecia, eyelash alopecia, intranasal hair alopecia, ophiasis pattern alopecia areata, sisaihpo pattern alopecia areata, male pattern hair loss, female pattern hair loss, anagen effluvium, telogen effluvium, hypotrichosis, hereditary hypotrichosis simplex, frontal fibrosing alopecia, cicatricial alopecia, lichen planopilaris, folliculitis decalvans, tufted folliculitis, dissecting cellulitis of the scalp, ring alopecia, chemotherapy induced alopecia, or superficial or deep infections of the scalp, or tinea capitis.

In certain embodiments, the compounds disclosed herein may be administered topically, that is by non-systemic administration. In certain embodiments, the compounds disclosed herein may be administered through systemic administration, including without limitation, oral, intravenous, subcutaneous, transdermal, intramuscular, intraperitoneal and intramuscular administration.

In certain embodiments, the compounds disclosed herein may be administered locally. In some embodiments, the inhibitors disclosed herein are topically administered to the skin overlying or in the proximity of the affected hair follicles. In some embodiments, the inhibitors disclosed herein are locally administered to the hair follicle by injection into or near the hair follicle. In some embodiments, the inhibitors disclosed herein are administered to the hair follicle. In certain embodiments, the inhibitor is administered to a subject's hair follicle. In certain embodiments, the inhibitor is administered to a subject's hair follicle when the hair follicle is in the telogen phase. In certain embodiments, the inhibitor is administered to a subject's hair follicle when the hair follicle is in the anagen or catagen phase.

In certain embodiments, the inhibitor is administered to a subject in such fashion as to deliver the inhibitor to the hair follicle, a part thereof, or a region near or around the hair follicle. In certain embodiments, the inhibitor is administered to a subject in such fashion as to deliver a therapeutically effective amount of the inhibitor to the hair follicle, a part thereof, or a region near or around the hair follicle. In certain embodiments, the inhibitor is administered to a subject in such fashion as to deliver a therapeutically effective amount of the inhibitor to the hair follicle, a part thereof, or a region near or around the hair follicle when the hair follicle is in the telogen phase.

In certain embodiments, the inhibitor is administered systemically. In some embodiments, the inhibitor is administered orally or by injection. In certain embodiments, the inhibitor is administered systemically when a subject's hair follicle is in the telogen phase. In certain embodiments, the inhibitor is administered systemically to a subject's hair follicle when the hair follicle is in the anagen or catagen phase.

In certain embodiments, the inhibitor is administered topically or orally. In certain other embodiments, particularly if the inhibitor is an antibody, it is administered by systemic or local injection.

In some embodiments, the inhibitor is administered locally, systemically, topically, orally, intradermally, intramuscularly, intraperitoneally, intravenously, subcutaneously, by intra-pulmonary administration, or by injection. In some embodiments, administration is to an alopecic area of the body. In some embodiments, administration is to a head, a scalp, a face, an eyebrow area, nasal hair area, or an eyelash area of the subject.

Also provided herein is a method of treating a hair loss disorder comprising administering to a patient in need thereof a therapeutically effective amount of an inhibitor of oncostatin, CSF1R, IL-34, or trichophage as disclosed herein, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

In certain embodiments, the therapeutically effective amount of an inhibitor of oncostatin, CSF1R, IL-34, or trichophage as disclosed herein, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof, may be in the form of a pharmaceutical composition.

In certain embodiments, the pharmaceutical composition may include a pharmaceutically acceptable excipient. In certain embodiments, the present disclosure is directed to methods of inducing hair growth in a subject in need thereof, the method comprising administering to the subject a therapeutically effective amount of a oncostatin, CSF1R, IL-34, and/or trichophage

In certain embodiments, the CSF1R inhibitor and/or a trichophage inhibitor is an antibody that specifically binds to a CSF1R protein or a fragment thereof; another trichophage protein or a fragment thereof, an antisense RNA, antisense DNA, an siRNA, an shRNA, a microRNA, or a variant or modification thereof that decreases expression of the gene that encodes the CSF1R protein or another trichophage-associated protein; an antisense RNA, antisense DNA, an siRNA, an shRNA, a microRNA, or a variant or modification thereof that decreases expression of the CSF1R protein or another trichophage-associated protein; a small molecule; or a combination thereof.

In some embodiments, the antibody may be selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), SNDX-6352 (an IgG4 humanized monoclonal antibody that binds to the ligand binding domain of the CSF-1 receptor, blocking the binding and consequent activation by both natural ligands (IL-34 and CSF-1)), or a combination thereof.

In some embodiments, the small molecule inhibitor that targets the CSF1R receptor may be selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

Methods for delivering the small molecule, the antisense RNA, antisense DNA, siRNA, shRNA, microRNA, or any variant or modification thereof can vary depending on the need and are well known to those skilled in the art. In certain embodiments, the components of a selected agent are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### Pharmaceutical Compositions and Administration

In certain embodiments, the CSF1R inhibitor and/or trichophage inhibitor compositions of the present disclosure can be formulated as pharmaceutical compositions or pharmaceutical formulations by admixture with a pharmaceutically acceptable carrier or excipient. In certain embodiments, the pharmaceutical formulations can include a therapeutically effective amount of a CSF1R inhibitor and/or trichophage inhibitor and a physiologically acceptable diluent or carrier. In certain embodiments, the pharmaceutical composition can further include one or more additional therapeutic components and/or adjuvants.

In certain embodiments, the pharmaceutical formulation can be a solid dosage form. In certain embodiments, the solid dosage form can be a tablet or capsule, cachets, pills, pellets, powders and granules.

In certain embodiments, the pharmaceutical formulation can be a liquid formulation. In certain embodiments, the liquid formulation can be an oral solution or oral suspension, topical solution, topical suspension, nanosuspension, fluid suspension, elixir.

In certain embodiments, the pharmaceutical formulation can be a topical dosage form which includes, but is not limited to, a spray, suppository, liniment, lotion, shampoo, solution, powder, fluid emulsion, suspension, nanoparticle, nanoparticle suspension, nanocapsule, liposomes, nanosuspension, fluid suspension, semi-solid, ointment, paste, cream, gel, jelly, foam or transdermal drug delivery system, e.g., a patch.

In certain embodiments, the pharmaceutical formulation can include liposomes, nanoparticles, and/or other carriers. In certain embodiments, the pharmaceutical formulation can include an adjuvant or enhancer, e.g., an enzyme inhibitor. In some embodiment, the pharmaceutical formulation can be administered in an extended release form, immediate release form, a delayed release form, a coated form, an enteric coated form, or combinations thereof.

In certain embodiments, the pharmaceutical formulation can be a direct infusion. In certain embodiments, the pharmaceutical formulation can be an implantable device.

Many methods can be used to introduce the formulations described herein, these include but are not limited to oral, topical, intradermal, intramuscular, intraperitoneal, intravenous, subcutaneous, and intra-pulmonary routes. All such routes are suitable for administration of these compositions and can be selected depending on the patient and condition treated if there is a condition present, and similar factors by an attending physician. According to the desired route for administration, the compositions of the disclosure are prepared in the form of, for example, liquids, powders, aerosols, tablets, capsules, enteric coated tablets or capsules, or suppositories.

Pharmaceutical compositions for topical administration may include foams, transdermal patches, ointments, lotions, creams, gels, solutions, fluid emulsions, fluid suspensions, semi-solids, pastes, drops, suppositories, sprays, liquids and powders. Formulations suitable for topical administration include liquid or semi-liquid preparations suitable for penetration through the skin to the target site such as a solution, powder, fluid emulsion, fluid suspension, semi-solid, ointment, paste, cream, gel, jelly, foam, liniment, lotion, spray, and drops suitable for administration to the eye, ear or nose.

The compositions can be formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferred unit dosage formulations are those containing an effective dose, as herein below recited, or an appropriate fraction thereof, of the active ingredient. The amount of active ingredient that may be combined with the carrier materials to produce a single dosage form will vary depending upon the host treated and the particular mode of administration.

It should be understood that in addition to the ingredients particularly mentioned above, the formulations described above may include other agents conventional in the art having regard to the type of formulation in question, for example those suitable for oral administration may include flavoring agents.

For preparing solid compositions such as tablets, the principal active ingredient can be mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally therapeutically effective unit dosage forms such as tablets, pills and capsules. This solid pre-formulation is then subdivided into unit dosage forms of the type described above containing from, for example, about 0.1 to about 1000 mg of the active ingredient.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Selection of the appropriate dosage for the priming compositions of the present disclosure can be based upon the physical condition of the mammal, most especially including the general health and weight of the mammal. Such selection and upward or downward adjustment of the effective dose is within the skill of the art.

In some embodiments, the inhibitor is administered at a dose from about 0.01% w/w to about 20% w/w. In some embodiments, for oral administration, the inhibitor is administered at a dose from about 0.0001 ug/kg body weight to about 40,000 ug/kg body weight.

In some embodiments, the inhibitor is administered in an amount of about 0.0001 μg/kg body weight, about 0.00025 μg/kg body weight, about 0.0005 μg/kg body weight, about 0.00075 μg/kg body weight, about 0.001 μg/kg body weight, about 0.0025 μg/kg body weight, about 0.005 μg/kg body weight, about 0.0075 μg/kg body weight, about 0.01 μg/kg body weight, about 0.025 μg/kg body weight, about 0.05 μg/kg body weight, about 0.075 μg/kg body weight, about 0.1 μg/kg body weight, about 0.25 μg/kg body weight, about 0.5 μg/kg body weight, about 0.75 μg/kg body weight, about 1 μg/kg body weight, about 5 μg/kg body weight, about 10 μg/kg body weight, about 25 μg/kg body weight, about 50 μg/kg body weight, about 75 μg/kg body weight, about 100 μg/kg body weight, about 150 μg/kg body weight, about 200 μg/kg body weight, about 250 μg/kg body weight, about 300 μg/kg body weight, about 350 μg/kg body weight, about 400 μg/kg body weight, about 450 μg/kg body weight, about 500 μg/kg body weight, about 550 μg/kg body weight, about 600 μg/kg body weight, about 650 μg/kg body weight, about 700 μg/kg body weight, about 750 μg/kg body weight, about 800 μg/kg body weight, about 850 μg/kg body weight, about 900 μg/kg body weight, about 950 μg/kg body weight, about 1000 μg/kg body weight, about 2000 μg/kg body weight, about 3000 μg/kg body weight, about 4000 μg/kg body weight, about 5000 μg/kg body weight, about 6000 μg/kg body weight, about 7000 μg/kg body weight, about 8000 μg/kg body weight, about 95000 μg/kg body weight, about 10,000 μg/kg body weight, about 15,000 μg/kg body weight, about 20,000 μg/kg body weight, about 40,000 μg/kg body weight or a range between any two of these values.

In some embodiments, the inhibitor is administered in an amount of about 0.1 μg/kg body weight to about 40,000 ug/kg body weight, about 0.1 μg/kg body weight to about 20,000 ug/kg body weight, about 0.1 μg/kg body weight to about 15,000 ug/kg body weight, about 0.1 μg/kg body weight to about 10,000 ug/kg body weight, about 0.1 μg/kg body weight to about 5,000 ug/kg body weight, about 0.1 μg/kg body weight to about 1,000 ug/kg body weight, about 0.1 μg/kg body weight to about 500ug/kg body weight, about 0.1 μg/kg body weight to about 100ug/kg body weight, about 0.1 μg/kg body weight to about 50ug/kg body weight, about 0.1 μg/kg body weight to about 10ug/kg body weight, about 0.1 μg/kg body weight to about 1 ug/kg body weight, about 1 μg/kg body weight to about 10,000 ug/kg body weight, about 1 μg/kg body weight to about 5,000 ug/kg body weight, about 1 μg/kg body weight to about 1,000 ug/kg body weight, about 1 μg/kg body weight to about 500ug/kg body weight, about 1 μg/kg body weight to about 100ug/kg body weight, about 1 μg/kg body weight to about 50ug/kg body weight, about 1 μg/kg body weight to about 10ug/kg body weight, about 10 μg/kg body weight to about 10,000 ug/kg body weight, about 10 μg/kg body weight to about 5,000 ug/kg body weight, about 10 μg/kg body weight to about 1,000 ug/kg body weight, about 10 μg/kg body weight to about 500ug/kg body weight, about 10 μg/kg body weight to about 100ug/kg body weight, about 10 μg/kg body weight to about 50ug/kg body weight, about 50 μg/kg body weight to about 10,000 ug/kg body weight, about 50 μg/kg body weight to about 5,000 ug/kg body weight, about 50 μg/kg body weight to about 1,000 ug/kg body weight, about 50 μg/kg body weight to about 500ug/kg body weight, about 50 μg/kg body weight to about 100ug/kg body weight, about 100 μg/kg body weight to about 10,000 ug/kg body weight, about 100 μg/kg body weight to about 5,000 ug/kg body weight, about 100 μg/kg body weight to about 1,000 ug/kg body weight, about 100 μg/kg body weight to about 500ug/kg body weight, about 500 μg/kg body weight to about 10,000 ug/kg body weight, about 500 μg/kg body weight to about 5,000 ug/kg body weight, about 500 μg/kg body weight to about 1,000 ug/kg body weight or a value within any of these ranges.

In some embodiments, the inhibitor is administered in an amount of about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 9.5 mg/kg body weight, about 10 mg/kg body weight, about 10.5 mg/kg body weight, about 11.0 mg/kg body weight, about 11.5 mg/kg body weight, about 12 mg/kg body weight, about 12.5 mg/kg body weight, about 13 mg/kg body weight, about 13.5 mg/kg body weight, about 14 mg/kg body weight, about 14.5 mg/kg body weight, about 15 mg/kg body weight, about 15.5 mg/kg body weight, about 16 mg/kg body weight, about 16.5 mg/kg body weight, about 17 mg/kg body weight, about 17.5 mg/kg body weight, about 18 mg/kg body weight, about 19.5 mg/kg body weight, about 20 mg/kg body weight, about 21.5 mg/kg body weight, about 22 mg/kg body weight, about 22.5 mg/kg body weight, about 23 mg/kg body weight, about 23.5 mg/kg body weight, about 24 mg/kg body weight, about 24.5 mg/kg body weight, about 25 mg/kg body weight, about 25.5 mg/kg body weight, about 26 mg/kg body weight, about 26.5 mg/kg body weight, about 27 mg/kg body weight, about 27.5 mg/kg body weight, about 28 mg/kg body weight, about 29.5 mg/kg body weight, about 30 mg/kg body weight, about 40 mg/kg body weight, or a value within any of these ranges.

In some embodiments, the inhibitor is administered in any of the following ranges: about 1 mg/kg body weight to about 50 mg/kg body weight, about 1 mg/kg body weight to about 40 mg/kg body weight, about 1 mg/kg body weight to about 30 mg/kg body weight, about 1 mg/kg body weight to about 20 mg/kg body weight, about 1 mg/kg body weight to about 10 mg/kg body weight, about 1 mg/kg body weight to about 5 mg/kg body weight, about 5 mg/kg body weight to about 50 mg/kg body weight, about 5 mg/kg body weight to about 40 mg/kg body weight, about 5 mg/kg body weight to about 30 mg/kg body weight, about 5 mg/kg body weight to about 20 mg/kg body weight, about 5 mg/kg body weight to about 10 mg/kg body weight, about 10 mg/kg body weight to about 50 mg/kg body weight, about 10 mg/kg body weight to about 40 mg/kg body weight, about 10 mg/kg body weight to about 30 mg/kg body weight, about 10 mg/kg body weight to about 20 mg/kg body weight, about 20 mg/kg body weight to about 50 mg/kg body weight, about 20 mg/kg body weight to about 40 mg/kg body weight, about 20 mg/kg body weight to about 30 mg/kg body weight, about 30 mg/kg body weight to about 50 mg/kg body weight, about 30 mg/kg body weight to about 40 mg/kg body weight, or a value within any of these ranges.

In some embodiments, the inhibitor is administered in an amount (in w/w %) of about 0.01%, about 0.05%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, or a range of any two of these two values.

In some embodiments, the inhibitor is administered in an amount (in w/w %) of about 0.01% to about 20%, about 0.05% to about 20%, about 0.1% to about 20%, about 0.2% to about 20%, about 0.3% to about 20%, about 0.4% to about 20%, about 0.5% to about 20%, about 0.6% to about 20%, about 0.7% to about 20%, about 0.8% to about 20%, about 0.9% to about 20%, about 1.0% to about 20%, 1.5% to about 20%, about 2.0% to about 20%, 2.5% to about 20%, about 3.0% to about 20%, about 4% to about 20%, about 5% to about 20%, about 6% to about 20%, about 7% to about 20%, about 8% to about 20%, about 9% to about 20%, about 0.01% to about 10%, about 0.05% to about 10%, about 0.1% to about 10%, about 0.2% to about 10%, about 0.3% to about 10%, about 0.4% to about 10%, about 0.5% to about 10%, about 0.6% to about 10%, about 0.7% to about 10%, about 0.8% to about 10%, about 0.9% to about 10%, about 1.0% to about 10%, 1.5% to about 10%, about 2.0% to about 10%, 2.5% to about 10%, about 3.0% to about 10%, about 4% to about 10%, about 5% to about 10%, about 6% to about 10%, about 7% to about 10%, about 8% to about 10%, about 9% to about 10%., about 0.1% to about 9%, about 0.1% to about 8%, about 0.1% to about 7%, about 0.1% to about 6%, about 0.1% to about 5%, about 0.1% to about 4%, about 0.1% to about 3%, about 0.1% to about 2.5%, about 0.1% to about 2%, about 0.1% to about 1.5%, about 0.1% to about 1%, about 0.1% to about 0.9%, 0.1% to about 0.8%, 0.1% to about 0.7%, 0.1% to about 0.6%, 0.1% to about 0.5%, 0.1% to about 0.4%, 0.1% to about 0.3%, 0.1% to about 0.2%., about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2.5%, about 0.5% to about 2%, about 0.5% to about 1.5%, about 0.5% to about 1%, about 0.5% to about 0.9%, 0.5% to about 0.8%, 0.5% to about 0.7%, 0.5% to about 0.6%, about 1% to about 9%, about 1% to about 9%, about 1% to about 8%, about 1% to about 7%, about 1% to about 6%, about 1% to about 5%, about 1% to about 4%, about 1% to about 3%, about 1% to about 2.5%, about 1% to about 2%, about 1% to about 1.5% of the composition, or a value within any of these ranges.

Pharmaceutical compositions of the present disclosure optionally further comprising sterile aqueous or non-aqueous solutions, suspensions, and emulsions. The composition can further comprise auxiliary agents or excipients, as known in the art. See, e.g., Berkow et al., eds., The Merck Manual, 15th edition, Merck and Co., Rahway, N.J. (2011); Goodman et al., eds., Modern Pharmaceutics, 5th Edition, Banker & Rhodes, CRC Press (2009); Goodman & Gilman's The Pharmaceutical Basis of Therapeutics, 12th Edition, McGraw Hill, N.Y. (2011); Avery's Drug Treatment: Principles and Practice of Clinical Pharmacology and Therapeutics, 3rd edition, ADIS Press, LTD., Williams and Wilkins, Baltimore, Md. (1987); Osol, A., ed., Remington's Pharmaceutical Sciences, Mack Publishing Co, Easton, Pa. pp. 1324-1341 (1980); Katzung, ed. Basic and Clinical Pharmacology, 14th Edition, McGraw-Hill Education, New York City, N.Y. (2017), which references and references cited therein, are entirely incorporated herein by reference as they show the state of the art.

In certain embodiments, preparations for parenteral administration include sterile aqueous or non-aqueous solutions, suspensions, and/or emulsions, which can contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance absorption. Liquid dosage forms for oral administration can generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents. See, e.g., Berkow, infra, Goodman, infra, Avery's, infra, Osol, infra and Katzung, infra, which are incorporated in their entirety herein by reference.

In certain embodiments, a composition of the present disclosure, used for administration to an individual, can further comprise salts, esters, deuterated derivatives, hydrates, polymorphs, stereoisomers, enantiomers, racemates, diastereomers, preservatives, chemical stabilizers, buffers, adjuvants, or other substances which are desirable for improving the efficacy of the composition. Typically, stabilizers, adjuvants, and preservatives are optimized to determine the best formulation for efficacy in the target human or animal. Suitable exemplary preservatives include chlorobutanol potassium sorbate, sorbic acid, sulfur dioxide, propyl gallate, the parabens, ethyl vanillin, glycerin, phenol, and parachlorophenol. Suitable stabilizing ingredients which can be used include, for example, casamino acids, sucrose, gelatin, phenol red, N—Z amine, monopotassium diphosphate, lactose, lactalbumin hydrolysate, and dried milk. Normally, the adjuvant and the composition are mixed prior to presentation, or presented separately, but into the same site of the mammal. Such adjuvants include, among others, MPL. (3-O-deacylated monophosphoryl lipid A; RIBI ImmunoChem Research, Inc., Hamilton, Mont.), mineral oil and water, aluminum hydroxide, Amphigen, Avridine, L121/squalene, D-lactide-polylactide/glycoside, pluronic plyois, muramyl dipeptide, killed Bordetella, saponins, such as Quil A or Stimulon QS-21 (Aquila Biopharmaceuticals, Inc., Framingham, Mass.) and cholera toxin (either in a wild-type or mutant form, e.g., wherein the glutamic acid at amino acid position 29 is replaced by another amino acid, preferably a histidine, in accordance with International Patent Application No. PCT/US99/22520, incorporated herein by reference). Additional examples of materials suitable for use in the compositions of the instant disclosure are provided in Osol, A., ed., Remington's Pharmaceutical Sciences, Mack Publishing Co, Easton, Pa. (1980), pp. 1324-1341, which reference is incorporated in its entirety herein by reference.

In some embodiments, the topical formulation of embodiments herein is administered in conjunction, concomitantly or adjunctively, with the therapies above and/or with a therapy for another disease. For example, the topical formulation of embodiments herein may be combined with thyroid hormone replacement therapy or with anti-inflammatory or immunomodulatory therapies.

### Methods of Monitoring Efficacy of Treatment

The present disclosure further relates to a method of assessing the efficacy of a therapy for promoting hair growth, inducing hair growth, maintaining the rate of hair growth, increasing the rate of hair growth, decreasing the rate of hair loss, preventing the onset or progression of a hair loss disorder, maintaining remission in a subject having a hair loss disorder, improving remission in a subject having a hair loss disorder, preventing hair loss, or the like in a mammalian subject. In certain embodiments, the method comprises (a) determining a level of one or more hair growth biomarkers in a hair follicle sample obtained from the subject, and (b) determining the level of the one or more biomarkers in a hair follicle sample obtained from the subject, at one of more time points during the therapy, wherein the therapy is efficacious for inducing or promoting hair growth in the subject when there is a change of the one or more biomarkers in the second or subsequent samples, relative to the first sample. In certain embodiments, the biomarkers are selected from Wnt pathway, Shh pathway, hair development pathway, melanogenesis pathway, or any combination thereof. In certain embodiments, the biomarkers are selected from the group consisting of CD34, Lhx2, NFATc1, Axin2, FoxC1, OSMR, OSM, Jak3, FAS, Irf1, Ifnar1, Nr3c1, Stat5A, I16st, Ptprc, Ghr, IL10ra, I12rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

The present disclosure further provides a method of assessing the efficacy of a therapy for promoting hair growth, inducing hair growth, maintaining the rate of hair growth, increasing the rate of hair growth, decreasing the rate of hair loss, preventing the onset or progression of a hair loss disorder, maintaining remission in a subject having a hair loss disorder, improving remission in a subject having a hair loss disorder, preventing hair loss, or the like in a mammalian subject by determining the level of CSF1R or the presence of a trichophage biomarker, such as TREM2, in the tissues surrounding a hair follicle sample obtained from the subject.

A biomarker can be a nucleic acid or a peptide/protein. Methods for qualitatively and quantitatively detecting and/or determining the expression level of a nucleic acid biomarker, include, but are not limited to polymerase chain reaction (PCR), including conventional, qPCR and digital PCR, RNA sequencing, single cell RNA sequencing, in situ hybridization (for example, but not limited to, Fluorescent In Situ Hybridization (“FISH”)), gel electrophoresis, sequencing and sequence analysis, microarray analysis and other techniques known in the art.

In certain embodiments, the method of detection can be real-time PCR (RT-PCR), quantitative PCR, fluorescent PCR, RT-MSP (RT methylation specific polymerase chain reaction), PicoGreen™ (Molecular Probes, Eugene, Oreg.) detection of DNA, radioimmunoassay or direct radio-labeling of DNA. For example, but not by way of limitation, a nucleic acid biomarker can be reversed transcribed into cDNA followed by polymerase chain reaction (RT-PCR); or, a single enzyme can be used for both steps as described in U.S. Pat. No. 5,322,770, or the biomarker can be reversed transcribed into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels of biomarker. The levels of a biomarker and a control mRNA can be quantitated in cancer tissue or cells and adjacent benign tissues. In certain embodiments, the levels of one or more biomarkers can be quantitated in a biological sample.

In a non-limiting embodiment, the method of detection of the present invention can be carried out without relying on amplification, e.g., without generating any copy or duplication of a target sequence, without involvement of any polymerase, or without the need for any thermal cycling. In certain embodiments, detection of the present invention can be performed using the principles set forth in the QuantiGene™ method described in U.S. application Ser. No. 11/471,025, filed Jun. 19, 2006, and is incorporated herein by reference.

In certain embodiments, in situ hybridization visualization can be employed, where a radioactively labeled antisense RNA probe is hybridized with a thin section of a biological sample, e.g., a biopsy sample, washed, cleaved with RNase and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

In certain non-limiting embodiments, evaluation of nucleic acid biomarker expression can be performed by fluorescent in situ hybridization (FISH). FISH is a technique that can directly identify a specific region of DNA or RNA in a cell and therefore enables visual determination of the biomarker expression in tissue samples. The FISH method has the advantages of a more objective scoring system and the presence of a built-in internal control consisting of the biomarker gene signals present in all non-neoplastic cells in the same sample. FISH is a direct in situ technique that can be relatively rapid and sensitive, and can also be automated. Immunohistochemistry can be combined with a FISH method when the expression level of the biomarker is difficult to determine by FISH alone.

In certain embodiments, expression of a nucleic acid biomarker can be detected on qPCR array, a DNA array, chip or a microarray. Oligonucleotides corresponding to the biomarker(s) are immobilized on a chip which is then hybridized with labeled nucleic acids of a biological sample, e.g., tumor sample, obtained from a subject. Positive hybridization signal is obtained with the sample containing biomarker transcripts. Methods of preparing DNA arrays and their use are well known in the art. (See, for example, U.S. Pat. Nos. 6,186,796; 6,379,897; 6,664,377; 6,451,536; 548,257; U.S. Patent Application Nos. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application No. 20030215858).

In certain embodiments, to monitor a nucleic acid biomarker, mRNA can be extracted from the biological sample to be tested, reverse transcribed and fluorescent-labeled cDNA probes can be generated. The labeled cDNA probes can then be applied to microarrays capable of hybridizing to a biomarker, allowing hybridization of the probe to microarray and scanning the slides to measure fluorescence intensity. This intensity correlates with the hybridization intensity and expression levels of the biomarker.

Types of probes for detection of nucleic acid biomarkers include cDNA, riboprobes, synthetic oligonucleotides and genomic probes. The type of probe used will generally be dictated by the particular situation, such as riboprobes for in situ hybridization, and cDNA for Northern blotting, for example. In certain non-limiting embodiments, the probe is directed to nucleotide regions unique to the particular biomarker RNA. The probes can be as short as is required to differentially recognize the particular biomarker mRNA transcripts, and can be as short as, for example, 15 bases. Probes of at least 17 bases, 18 bases and 20 bases can also be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, 32P and 35S, or fluorophores. Labeling with radioisotopes can be achieved, whether the probe is synthesized chemically or biologically, by the use of suitably labeled bases.

Methods for detecting and/or determining the level of a protein biomarker are well known to those skilled in the art, and include, but are not limited to, mass spectrometry techniques, 1-D or 2-D gel-based analysis systems, chromatography, enzyme linked immunosorbant assays (ELISAs), radioimmunoassays (RIA), enzyme immunoassays (EIA), Western Blotting, immunoprecipitation and immunohistochemistry. These methods use antibodies, or antibody equivalents, to detect protein, or use biophysical techniques. Antibody arrays or protein chips can also be employed, see, for example, U.S. Patent Application Nos. 2003/0013208; 2002/0155493, 2003/0017515 and U.S. Pat. Nos. 6,329,209 and 6,365,418, herein incorporated by reference in their entireties.

In certain non-limiting embodiments, a detection method for measuring protein biomarker expression includes the steps of: contacting a biological sample, e.g., a tissue sample, with an antibody or variant (e.g., fragment) thereof, which selectively binds the biomarker, and detecting whether the antibody or variant thereof is bound to the sample. The method can further include contacting the sample with a second antibody, e.g., a labeled antibody. The method can further include one or more washing steps, e.g., to remove one or more reagents.

In certain non-limiting embodiments, Western blotting can be used for detecting and quantitating biomarker protein expression levels. Cells can be homogenized in lysis buffer to form a lysate and then subjected to SDS-PAGE and blotting to a membrane, such as a nitrocellulose filter. Antibodies (unlabeled) can then brought into contact with the membrane and assayed by a secondary immunological reagent, such as labeled protein A or anti-immunoglobulin (suitable labels including 125I, horseradish peroxidase and alkaline phosphatase). Chromatographic detection can also be used. In certain embodiments, immunodetection can be performed with antibody to a biomarker using the enhanced chemiluminescence system (e.g., from PerkinElmer Life Sciences, Boston, Mass.). The membrane can then be stripped and re-blotted with a control antibody specific to a control protein, e.g., actin.

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled, antibody. Labeling can be by fluorescent markers, enzymes, such as peroxidase, avidin or radiolabeling. The assay can be scored visually, using microscopy, and the results can be quantitated. Machine-based or autoimaging systems can also be used to measure immunostaining results for the biomarker.

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Labeled antibodies against biomarkers can also be used for imaging purposes, for example, to detect the presence of a biomarker in cells of a subject. Suitable labels include radioisotopes, iodine (125I, 121I), carbon (14C), sulphur (35S), tritium (3H), indium (112In), and technetium (99mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red. The labeled antibody or antibody fragment will preferentially accumulate at the location of cells which contain a biomarker. The labeled antibody or variant thereof, e.g., antibody fragment, can then be detected using known techniques.

Antibodies include any antibody, whether natural or synthetic, full length or a fragment thereof, monoclonal or polyclonal, that binds sufficiently strongly and specifically to the biomarker to be detected. An antibody can have a Kd of at most about 10-6M, 10-7M, 10-8M, 10-9M, 10-10M, 10-11M and 10-12M. The phrase “specifically binds” refers to binding of, for example, an antibody to an epitope or antigen or antigenic determinant in such a manner that binding can be displaced or competed with a second preparation of identical or similar epitope, antigen or antigenic determinant.

Antibodies, and derivatives thereof, that can be used encompass polyclonal or monoclonal antibodies, synthetic and engineered antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a biomarker, or portions thereof, including, but not limited to, Fv, Fab, Fab′ and F(ab′)2 fragments, can be used. Such fragments can be produced by enzymatic cleavage or by recombinant techniques.

In certain non-limiting embodiments, agents that specifically bind to a polypeptide other than antibodies are used, such as peptides. Peptides that specifically bind can be identified by any means known in the art, e.g., peptide phage display libraries. Generally, an agent that is capable of detecting a biomarker polypeptide, such that the presence of a biomarker is detected and/or quantitated, can be used. As defined herein, an “agent” refers to a substance that is capable of identifying or detecting a biomarker in a biological sample (e.g., identifies or detects the mRNA of a biomarker, the DNA of a biomarker, the protein of a biomarker).

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos. 2003/0199001, 2003/0134304, 2003/0077616, which are herein incorporated by reference in their entireties.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

Detection of the presence of a biomarker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis), to determine the relative amounts of a particular biomarker. Software programs such as the Biomarker Wizard program (Ciphergen Biosystems, Inc., Fremont, Calif.) can be used to aid in analyzing mass spectra.

Additional methods for determining nucleic acid and/or protein biomarker expression in samples are described, for example, in U.S. Pat. Nos. 6,271,002; 6,218,122; 6,218,114; and 6,004,755; and in Wang et al, J. Clin. Oncol., 22(9): 1564-1671 (2004); and Schena et al, Science, 270:467-470 (1995); all of which are incorporated herein by reference in their entireties.

### Kits

The present disclosure further relates to a kit for treating a hair loss disorder, promoting hair growth, inducing hair growth, maintaining the rate of hair growth, increasing the rate of hair growth, decreasing the rate of hair loss, preventing the onset or progression of a hair loss disorder, maintaining remission in a subject having a hair loss disorder, improving remission in a subject having a hair loss disorder, preventing hair loss, or the like in a mammalian subject. In certain embodiments, the kit comprises (a) a CSF1R inhibitor, IL-34 inhibitor, oncostatin inhibitor, and/or trichophage inhibitor; and (b) a pharmaceutically acceptable carrier.

In certain embodiments, the CSF1R inhibitor, IL-34 inhibitor, oncostatin inhibitor, and/or trichophage inhibitor is an antibody that specifically binds to a CSF1R protein or a fragment thereof; a trichophage-associated protein or a fragment thereof; an antisense RNA, antisense DNA, an siRNA, an shRNA, a microRNA, or a variant or modification thereof that decreases expression of the gene that encodes the CSF1R protein or another trichophage-associated protein; an antisense RNA, antisense DNA, an siRNA, an shRNA, a microRNA, or a variant or modification thereof that decreases expression of the CSF1R protein or another trichophage-associated protein; a small molecule; or a combination thereof.

In some embodiments the antibody may be selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), SNDX-6352 (an IgG4 humanized monoclonal antibody that binds to the ligand binding domain of the CSF-1 receptor, blocking the binding and consequent activation by both natural ligands (IL-34 and CSF-1)), or a combination thereof.

In some embodiments, the small molecule inhibitor that targets the CSF1R receptor may be selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

In some embodiments, the kit further comprises an applicator, instructions for use, or a combination thereof.

### EXAMPLES

The following examples are put forth so as to provide those of ordinary skill in the art with a complete disclosure and description of how to make and use the presently disclosed subject matter, including, but not limited to compositions and methods for the induction of hair growth by inhibition of the JAK-STAT pathway, and particularly inhibition of CSF1R and/or trichophages, in order to induce hair growth. The following examples are not intended to limit the scope of the presently disclosed subject matter. It is understood that various other embodiments may be practiced, given the general description provided above.

The following materials and methods were used in these examples.

### Animals

Mice were bred and maintained in the Russ Berrie Medical Sciences Pavilion Animal Facility in accordance with guidelines of the Institute of Comparative Medicine (ICM) and Institutional Animal Care and Use Committee (IACUC) of Columbia University. The facility is specific pathogen-free, and all mice were socially housed under a 12 hour light/dark cycle. All experiments were performed during the early- to mid-telogen phase of the hair cycle (P45-P60), unless otherwise specified. K5-CreERT2::OSMRβFL/FL or K5-CreERT2::STAT5a/bFL/FL were compared with control littermates (No CreER or OSMRβwt/wt/STAT5a/bwt/wt). For genetic ablation of macrophages, CSF1R-CreER::R26-iDTR mice were compared with control WT littermates (no CreER). K17-CreERT2 mice were generously provided by Dr. David Owens. CSF1R-CreER mice were generously provided by Dr. Tony Ferrante, and R26R-iDTR mice were provided by Dr. David Owens.

### Tamoxifen Induction

Tamoxifen (Sigma-Aldrich) was dissolved in corn oil to a concentration of 10 mg/ml, and mice were injected intraperitoneally (200 μl) under light-protected conditions for 4 consecutive days.

### EdU

5-ethnyl-2′-deoxyuridine (EdU, Life Technologies) was dissolved in sterile PBS to a concentration of 10 mg/ml. For cellular dynamic studies, a single dose of 50 μg/g was injected intraperitoneally 24 hours prior to sacrifice.

### Hair Cycle Manipulation

Mice were carefully shaved with clippers during telogen to reveal the pink skin typical of the telogen phase 1 week prior to the experiment. Mice that were inadvertently wounded were not used. Anagen was induced by topical application of 2% Ruxolitinib in DMSO to the right side of the dorsal skin daily for 5 consecutive days. The hair cycle was observed and documented with standardized photographs taken prior to the first treatment, and then twice weekly thereafter. Murine IL-6, LIF or OSM (100 ng/ml, 50 ng/ml, 125 ng/ml respectively) were dissolved in sterile PBS and 100 μl was injected into the center of the field of application daily for 10 consecutive days, beginning with the first application of Ruxolitinib.

### Neutralizing Antibodies

Neutralizing antibodies to OSMR (7.5 μg) were injected intradermally into the dorsal telogen skin daily for 14 days from P60 (mid-telogen). Neutralizing antibodies to CSFL1R (AFS98) and F4/80 (CI:A3-1) were diluted in sterile PBS, and 500 μg was injected intradermally into the dorsal telogen skin for 14 consecutive days. Neutralizing antibodies to CD25 (PC61.5) were injected intraperitoneally every other day (250 μg/dose×3) from P53, prior to anagen induction at P60.

### Flow Cytometry

Dorsal skin was processed for either epidermal or dermal single-cell suspensions for stem cell or immune cell analysis by flow cytometry. Full thickness skin was harvested and defatted, and floated on 0.25% Trypsin for 30 min at 37° C. Epidermal cells were scraped off and titurated in DMEM/10% FBS before filtering through a 70 μm mesh and centrifuged to obtain the epidermal cell pellet. The dermis was macerated finely with dissection scissors and re-suspended in 5 ml DMEM with 0.2% Collagenase IV and 300U DNase 1, and incubated in a 37° C. water bath for 40 minutes. The digested dermis was titurated in DMEM/10% FBS and filtered through a 70 μm mesh and centrifuged. Cells were labelled with conjugated surface antibodies listed in the Key Resources Table in DMEM/2% FBS for 1 h on ice, and washed and labelled with live/dead Hoescht stain prior to FACS. Flow cytometry was performed on the Influx sorter in the Columbia University Flow Cytometry Core. Epidermal cell suspensions were used for HFSC stem cell analysis, and dermal suspensions were sorted for dermal papilla and immune cell experiments. Cells were collected in DMEM/10% FBS for cell culture, or in Trizol for RNA extraction. Flow cytometry data was analyzed using FlowJo software (FlowJo, LLC).

### Cell Culture

HFSCs (ITGA6+ Sca-1−) cells were collected from flow cytometry and plated onto 6-well plates, and maintained with Cnt-07S keratinocyte media. In vitro stimulation experiments were carried out when keratinocytes were 80-90% confluent. Confluent HFSCs were pre-treated with JAK inhibitors (tofacitinib, ruxolitinib or PF-06651600, all at 10 μM) for 20 min, and murine OSM was added for a final concentration of 10 ng/ml or 20 ng/ml for 15 min. For clonogenic assays, HFSCs were plated at a density of 10,000 cells per well and maintained for 2 weeks. For analysis of clonogenicity, plates were washed with PBS, and keratinocytes were fixed in situ with 4% paraformaldehyde (PFA) for 1 hour, followed by staining with 1% (wt/vol) Rhodamine B (Sigma-Aldrich) for 1 h. Clones were quantified using a backlight.

qRT-PCR

Sorted cell populations, epidermal sheets or dermal tissue was collected in Trizol and flash-frozen overnight at −80° C. RNA was extracted with the QIAgen RNeasy Micro Kit and cDNA was made using Superscript IV with a 2:1 mixture of random hexamers and oligo-dT primers. Semi-quantitative PCR for genes listed in the Key Resources Table was performed using SYBR Green PCR mix on an Applied Biosystems 7300 Real-Time PCR System. Primers for GAPDH were used in each reaction as a housekeeping control, and fold changes were calculated using the δ-δ Ct algorithm. Error bars were calculated based on SD across three biological replicates. An unpaired two-tailed t-test was used to calculate significance between samples.

### Western Blot

Cells were lyzed in RIPA buffer in the presence of protease and phosphatase inhibitors on ice, and protein lysates were resuspended in Laemmli sample buffer. Whole-cell lysates were fractionated on TGX Stain-free protein gels and transferred to a PVDF membrane, blocked with 5% non-fat milk in TBST, and incubated with antibodies listed in the Key Resources Table (all 1:1000, diluted in TBST/3% BSA) overnight. Membranes were washed the following day and incubated with HRP-conjugated secondary antibodies (1:5000), washed, and developed with Luminata Forte Western HRP Substrate and visualized on the BioRAD ChemiDOC MP Imaging system. GAPDH or (3-actin were used as loading controls, depending on the mass of the proteins of interest.

### Immunofluorescence and Histology

For immunofluorescence (IF) studies, dorsal skin or human scalp biopsies were submerged in 4% PFA for 1 hour, washed in PBS, and allowed to sink in 30% sucrose overnight before being embedded and frozen in OCT over liquid nitrogen. Samples were sectioned at 8 μm thickness onto SuperFrost Plus glass slides (Fisher Scientific), blocked with 2% fish skin gelatin in PBS/0.3% Triton-X, and labelled with primary antibodies listed in Key Resources Table overnight at 4° C. Primary antibodies were washed off the following day, and labelled with fluorescence-conjugated secondary antibodies (1:1000), and images were acquired on a Zeiss LSM 5 Exciter Confocal microscope. EdU labelling was carried out with Click-iT Plus Alexa Fluor 647 nm Imaging Kit according to manufacturer's protocol. For histology, formalin-fixed paraffin-embedded (FFPE) sections of mouse dorsal skin were rehydrated in increasingly dilute ethanol, and stained with hematoxylin and eosin.

shRNA

Hairpin sequences containing scramble or OSM 21mers obtained from the TRC RNAi consortium were cloned into the pLKO.1 library vector between the AgeI and EcoRI restriction sites. Modified pLKO.1 vector, along with helper packaging plasmids pMD2.G and pCMVδR8.2, were transfected into 293FT cells in the presence of Lipofectamine 3000 reagent, used according to manufacturer's protocol. Supernatant containing lentivirus was harvested 48 hours after transfection, filtered through a 0.45 μm syringe filter, concentrated with PEG-it viral precipitant, resuspended in sterile PBS, and stored at −80° C. until required.

### Macrophage Inhibition

For in vivo macrophage inhibition, the small molecule Pexidartinib/PLX3397 was administered topically (2 mM in DMSO) or subcutaneously (1 mM in corn oil) for 5 consecutive days from P60. For OSM knock-down, peritoneal macrophages were harvested from adult C57BL/6 mice and cultured in DMEM/F12/15% FBS/0.3 mMCa2+ the presence of 0.1 mg/ml M-CSF to polarize them to an “M2-like” phenotype, similar to trichophages. Lentiviral precipitate containing scrambled or OSM shRNA added to the media in the presence of 8 μg/ml protamine sulphate, and the cells were centrifuged at 3000 rpm for 1 h at 32° C. to enhance transduction. Successful transductions were enriched with puromycin (1 μg/ml) selection, counted and used for the patch assay.

### Hair Reconstitution Assays

Neonatal (P0 or P1) mice were sacrificed and skins were harvested for the patch assay. Neonatal skins were enzymatically separated with 0.25% trypsin, and the dermis was further digested with 0.3% collagenase/DNase. Neonatal keratinocytes and dermal cells were counted recombined in a 1:1 ratio (106 cells each) and resuspended in 100 μl of media (1:1 mix of DMEM/10% FBS and CnT-0.7S) and injected intradermally into the dorsal skin of a nude mouse. For pharmacological/cytokine treatment, tofacitinib (400 nM) and/or OSM (25 ng/ml) was added. For macrophage inhibition assays, 20×103 macrophages (either freshly sorted by FACS, or cultured) were added to the cell slurry before injection into the Foxn1nu-2J nude mouse.

### Single-Cell RNA Sequencing

Live CD45+ immune cells from the dermis was isolated by FACS at early, mid and late telogen, captured on a microfluidic chip, and processed for single-cell RNA sequencing with the 10× Genomics Chromium 3′ Solution platform. cDNA synthesized by this method was amplified and sequenced on an Illumina NextSeq. Cells with <500 or >2000 genes were excluded from analysis, as were cells with >105 UMIs (reflecting doublets) or mitochondrial genes >0.8% (reflecting dying cells). 1186 highly variable genes were identified based on their average expression and variance, and used for clustering analysis. Principle component analysis (PCA) on variable genes was performed, and tSNE was run on 12 PCAs. Further analysis and presentation of data was performed with the Seurat R package (Satija Lab).

### Example 1: OSM Maintains Hair Follicles in Telogen Via OSMR-JAK-STAT Signaling

Data in this example and related figures are mean±SEM. *p<0.05, **p<0.01, ***p<0.001, ****p<0.0001, Student's unpaired t-test.

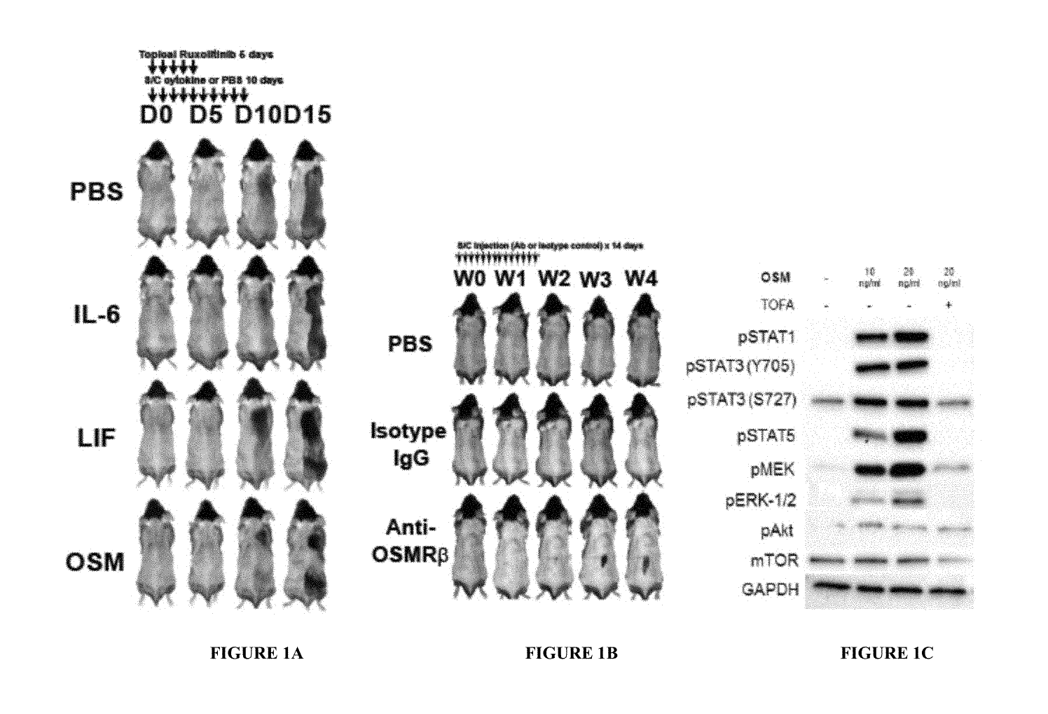

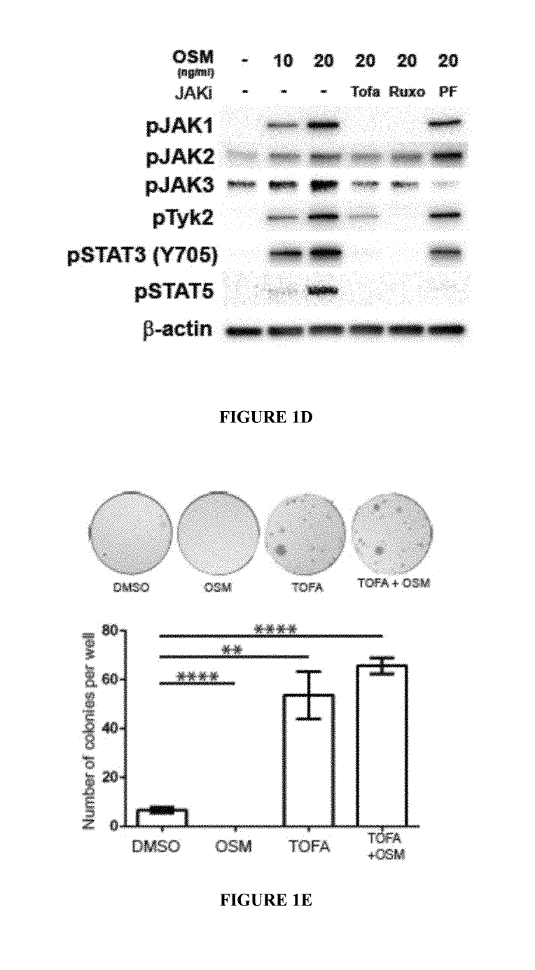

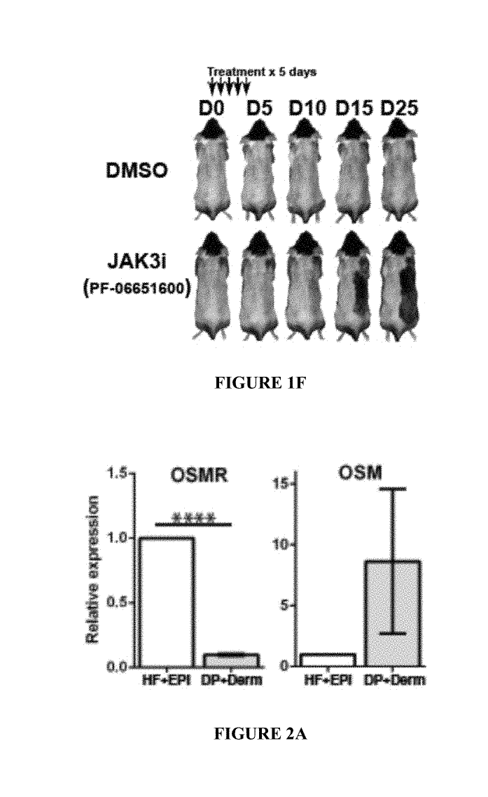

OSM and other gp130 cytokines (IL-6 and Leukemia Inhibitory Factor, LIF) were tested for their ability to inhibit anagen resulting from topical JAK inhibitors, which resembles spontaneous anagen. C57BL/6 mice were treated on the right dorsal skin topically with Ruxolitinib 2% in DMSO at P60 (mid-telogen) for 5 days, recapitulating previous experiments in Harel, S., et al., Pharmacologic inhibition of JAK-STAT signaling promotes hair growth. Sci Adv, 2015. 1(9): p. e1500973. This treatment regimen stimulated a robust anagen in the telogen skin that resembled the spontaneous, normal hair cycle. From P60, mice were also injected with control PBS or a gp130 cytokine (IL-6, LIF or OSM) into the middle of the field of topical application for 10 days. Intradermal OSM was the only member of this class of cytokines that inhibited anagen in the area it was administered. (FIG. 1A).

Conversely, intradermal injection of neutralizing antibodies to the OSM receptor (OSMRβ) into the dorsal telogen skin (P60) of C57BL/6 mice for 2 weeks produced the complementary effect of local anagen induction. This effect was not observed with intradermal PBS or isotype IgG control. (FIG. 1B).

Hair follicle stem cells (HFSCs) (ITGA6+ Sca-1− population isolated from telogen skin), were cultured and stimulated with OSM at 10 ng/ml and 20 ng/ml. Western blots performed on cultured HFSCs showed that OSM activated JAK-STAT1/3/5 pathways and the MAPK-MEK-ERK pathways in HFSCs in a dose-dependent fashion, and had minimal effect on the PI3K/Akt/mTOR pathway. Pre-treatment with Tofacitinib inhibited the JAK-STAT and, to a lesser extent, the MAPK-MEK-ERK pathways. (FIG. 1C).

A Western blot of cultured HFSCs stimulated with OSM showed that OSM exhibited a dose-dependent activation of all four JAKs (JAK1, JAK2, JAK3, Tyk2), as well as pSTAT3 and pSTAT5. Tofacinitib and Ruxolitinib inhibited phosphorylation of a variety of JAKs, and inhibited both pSTAT3 and pSTAT5 activation. PF-06651660, a specific covalent inhibitor of JAK3, did not have an effect on JAK1/JAK2/Tyk2 or STAT3 phosphorylation but still inhibited pSTAT5 activation. (FIG. 1D).

Notably, the covalent JAK3 inhibitor PF-06651600 was able to initiate anagen with the 5-day topical treatment regimen previously described (FIG. 1F), without significant inhibition of STAT3 phosphorylation. This suggests that inhibition of STAT5 phosphorylation, and not STAT3, in HFSCs is the common mechanism, and is sufficient to promote anagen initiation in mid-telogen skin.

Colony-forming assays with isolated (ITGA6+ Sca-1−) HFSCs were performed in the presence of OSM (10 ng/ml), Tofacitinib (100 nM) or both. OSM prevented colonies from forming in vitro, consistent with its inhibitory effect on HFSC proliferation. Tofacitinib increased the colony-forming ability of HFSCs, as well as increasing the colony size, consistent with previous reports (Doles, J., et al., Age-associated inflammation inhibits epidermal stem cell function. Genes Dev, 2012. 26(19): p. 2144-53). The addition of Tofacitinib was also sufficient to rescue the inhibitory effects of OSM. (FIG. 1E). These results suggest that OSM acts via JAK-STAT signaling, and most likely via activation of STAT5, to prevent proliferation of HFSCs, and is sufficient for maintaining HFSC quiescence during telogen in mouse skin.

### Example 2: OSMRβ and its Co-Receptor Gp130 are Expressed on Bulge and Germ HFSCs, Co-Localizing with Activated pSTAT5 During Early and Mid-Telogen

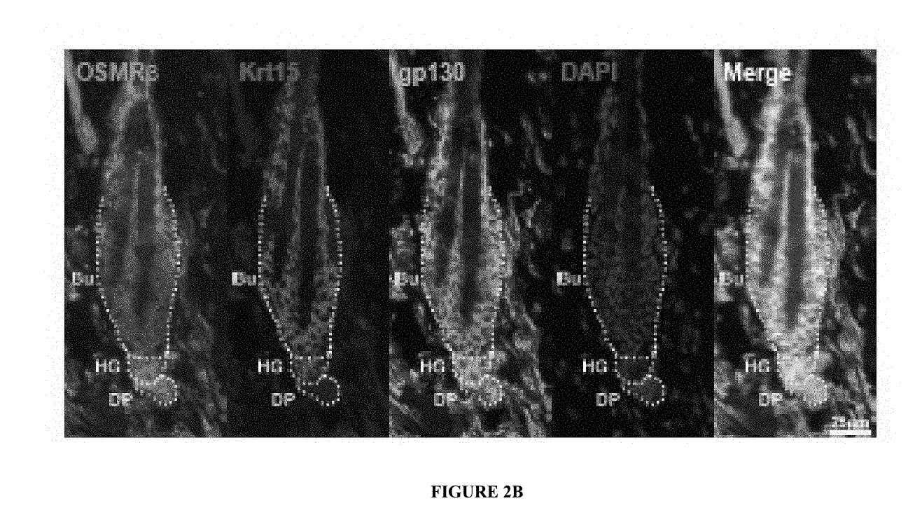

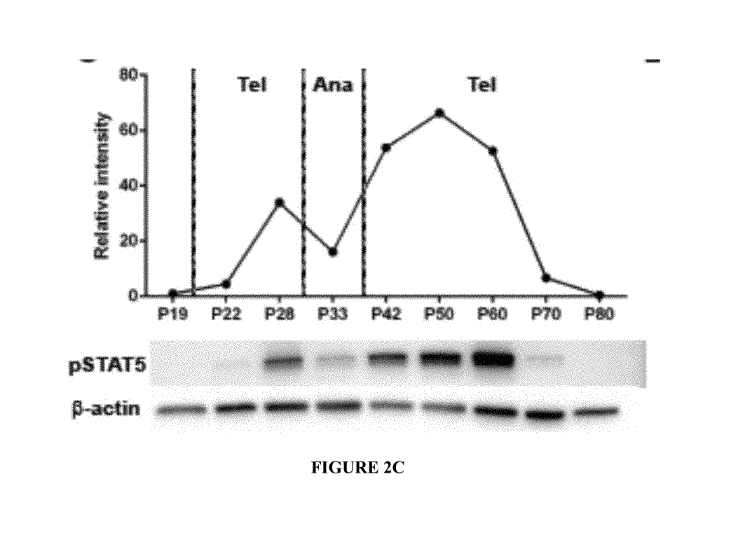

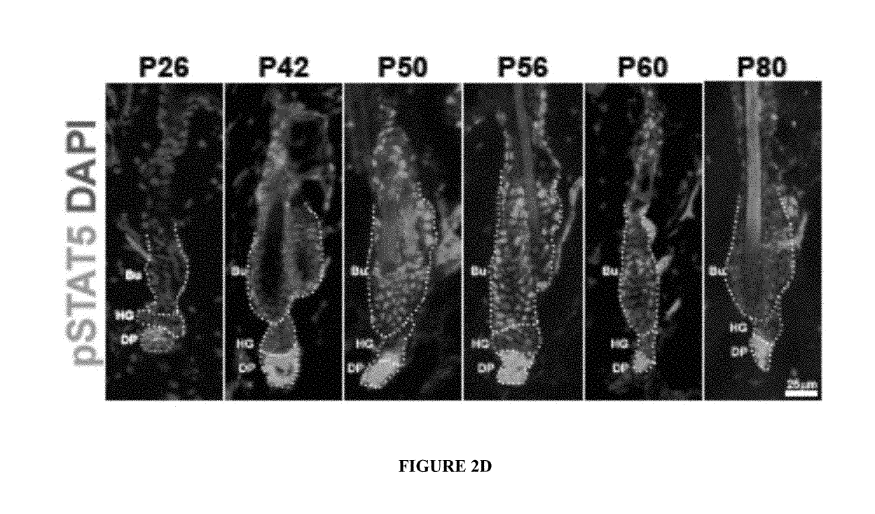

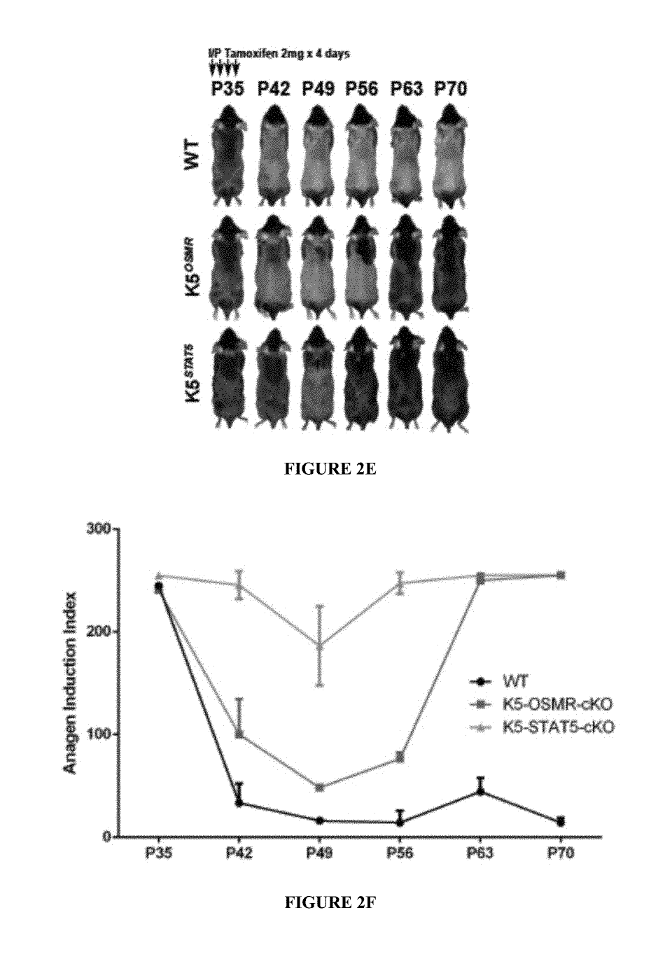

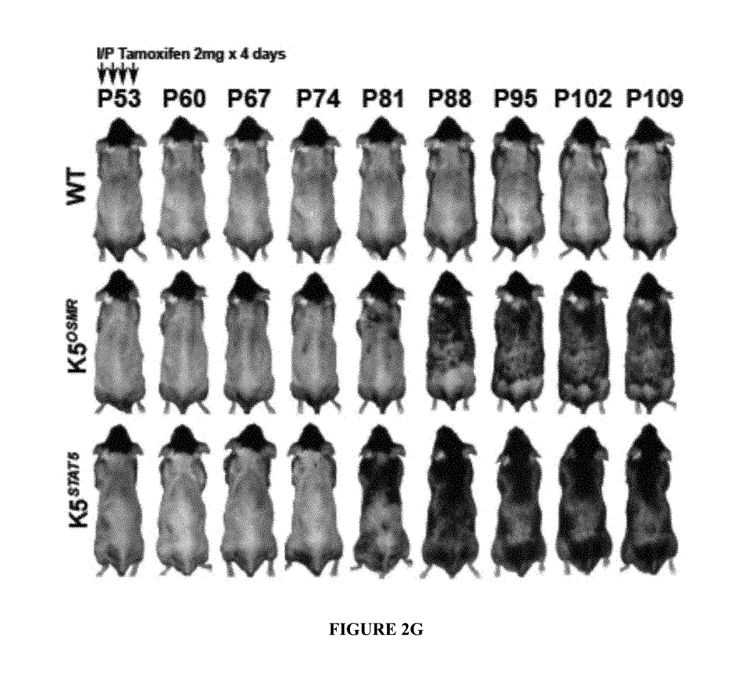

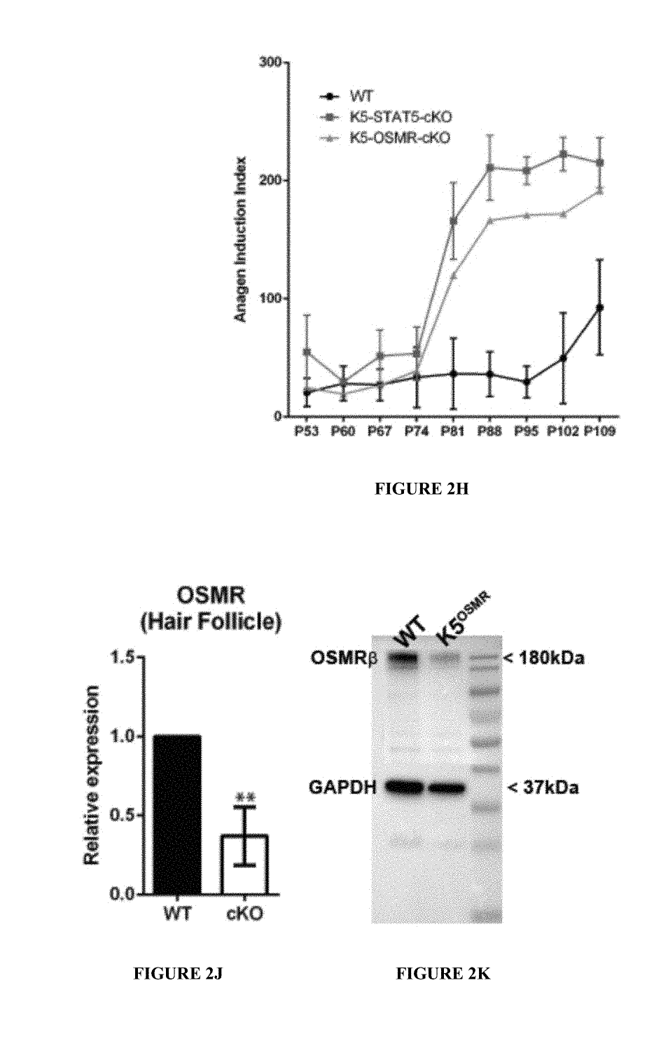

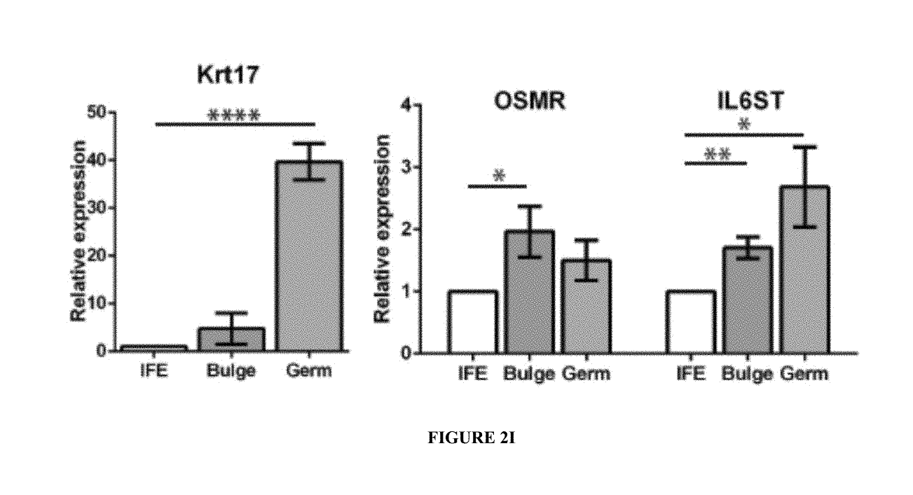

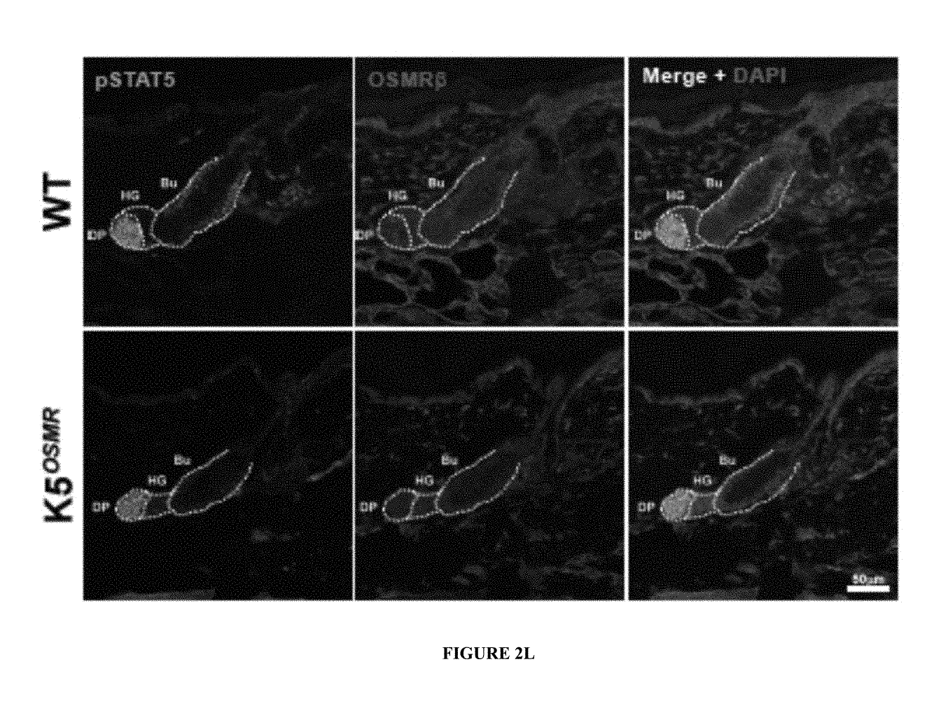

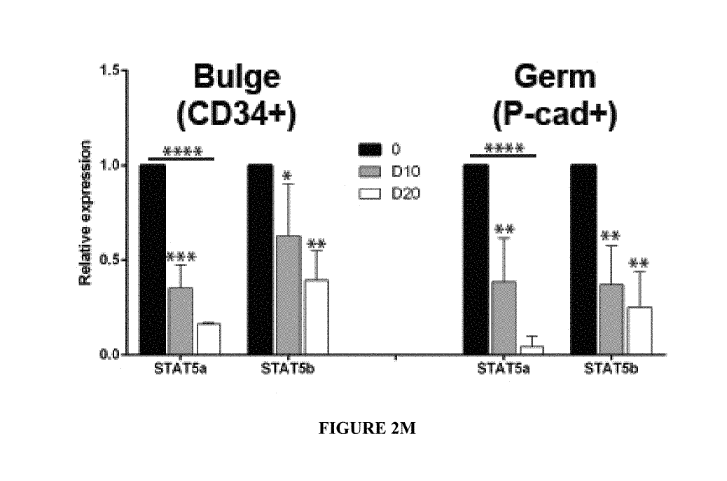

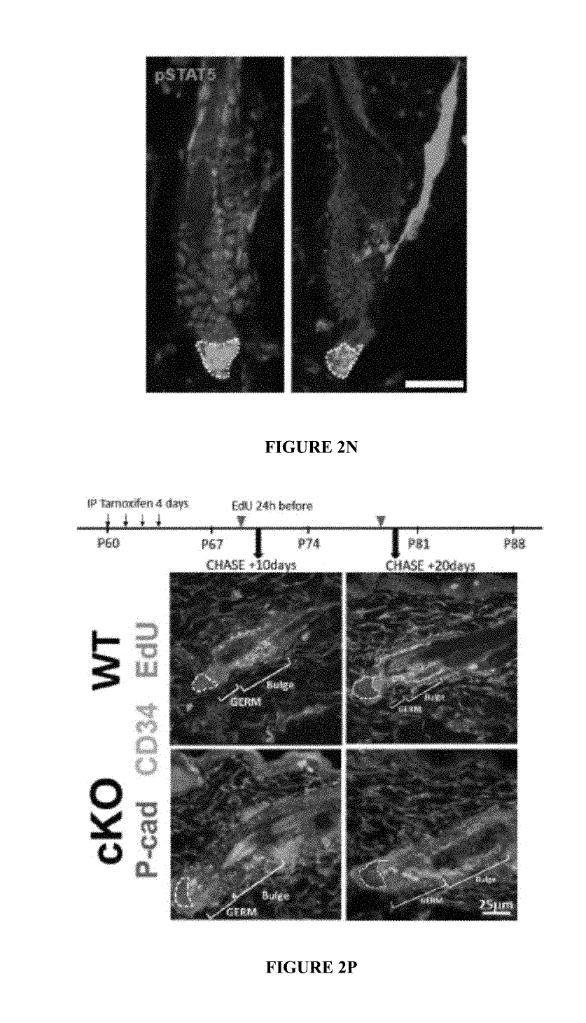

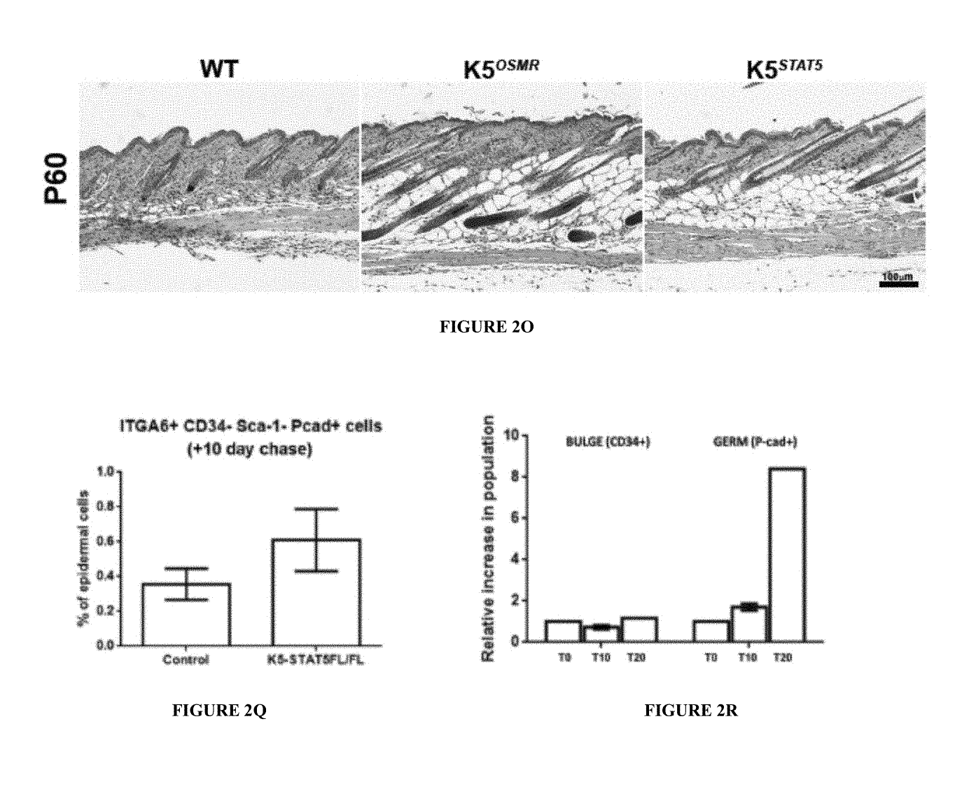

In the mouse, OSM binds specifically to its receptor OSMR, which requires the co-receptor gp130 for signaling via the JAK-STAT pathway. The expression of OSM, its receptor (OSMRβ) and co-receptor (gp130) in hair follicles was further investigated. By separating the mouse dorsal skin from P60 C57BL/6 mice into the epidermal and dermal fractions with 0.25% trypsin and using qRT-PCR for OSMRβ and OSM, it was demonstrated that OSMRβ was distinctly expressed in the epidermal fraction (which contains the epithelial parts of hair follicles) (p<0.0001), whereas OSM was located in the dermal fraction (which contains the dermal cells and the dermal papilla, DP) OSM (p=0.21). A clear dermal-epidermal signaling axis for OSM was also demonstrated. (FIG. 2A).

To further define the expression of OSMRβ and gp130, the epidermal fraction was sorted by flow cytometry (FACS) into the Sca-1+ interfollicular epidermis (IFE), CD34+ bulge, and the P-cadherin+ hair germ (HG). qRT-PCR for Keratin 17 (hair shaft keratin) was performed to confirm the sorting strategy. OSMRβ and gp130 are expressed at higher levels in the HFSCs of the bulge and HG, with an increasing expression of Krt17 from IFE->Bulge->HG. (FIG. 2I). qRT-PCR confirmed that OSMRβ and gp130 were preferentially expressed in the bulge and HG of telogen HFSCs. This was confirmed with immunofluorescence of P60 telogen hair follicle showing co-localization of OSMRβ and gp130 in the HFSC compartment, marked with the stem cell marker Keratin 15. gp130 expression was ubiquitous, but was strong in the HG. OSMRβ was also expressed in scattered dermal cells. (FIG. 2B; scale bar 25 μm).

In keeping with previous qRT-PCT array data of Harel, 2015, showing dynamic STAT5 activity over telogen, Western blotting of isolated epidermal sheets showed that activated pSTAT5 protein was also dynamically expressed in the epidermis across telogen, with expression prominent during early-to-mid second telogen (P42-P60), peaking at mid-telogen (P56-P60). (FIG. 2C; Tel=telogen, Ana=anagen).

Using immunofluorescence studies of pSTAT5 across the hair cycle, it was demonstrated that pSTAT5 during early- and mid-telogen is localized prominently in the bulge and HG HFSCs. In particular, pSTAT5 localizes to the HFSC compartment particularly strongly from P42-P60, which represents early-to-mid second telogen, and is diminished in the first telogen (P26) and late second telogen (P80), when the hair follicle is ready to enter the next anagen phase. (FIG. 2D). pSTAT5 expression in the HFSC decreases by the end of telogen (P80)(Flores, A., et al., Lactate dehydrogenase activity drives hair follicle stem cell activation. Nat Cell Biol, 2017).

### Example 3: Genetic Ablation of OSMRβ Before Early Telogen Delays and Shortens the Second Telogen

To examine the significance of OSMRβ and pSTAT5 localization to telogen hair follicles, C57BL/6 mice with K5-CreERT₂ transgenes coupled with OSMRβ^{FL/FL} or STAT5a/b^{FL/FL} alleles were generated. Mice were closely shaved with clippers to allow for direct visualization of the dorsal skin. Systemic administration of tamoxifen was carried out during early-mid telogen to genetically ablate OSMRβ or STAT5 in epidermal stem cells, including the HFSC.

OSMRβ and STAT5 conditional knock-out in K5-CreERT₂::OSMRβ^{FL/FL} mice was confirmed with qRT-PCR, Western blot and immunofluorescence studies. (FIGS. 2J-2N). In these studies, data are mean±SEM. *p<0.05, **p<0.01, ***p<0.001, ****p<0.0001. These data show that elevated OSMRβ-pSTAT5 signaling in HFSCs is correlated with early- and mid-telogen, and decreases during late telogen.

In FIG. 2J, data from qRT-PCR for OSMRβ in the hair follicle of K5-CreERT₂::OSMRβ^{FL/FL} mice is presented. HFSCs include bulge and HG cells (ITGA6+ Sca-1−).

In FIG. 2K, a Western blot of whole epidermis in K5-CreERT₂::OSMRβ^{FL/FL} mice showing reduced expression of OSMRβ after Tamoxifen induction is presented.

In FIG. 2L, results from immunofluorescence studies of telogen HFs in WT and K5-CreERT₂::OSMRβ^{FL/FL} mice are presented. K5-CreERT₂::OSMRβ^{FL/FL} mice had reduced expression of OSMRβ in the bulge and HG, coupled with reduced pSTAT5 activity in the HFSCs. pSTAT5 expression in the DP remained unchanged. Interestingly, epidermal OSMRβ ablation was associated with loss of pSTAT5 in the HFSCs.

In FIG. 2M, data from qRT-PCR for STAT5a and STATb in K5-CreERT₂::STAT5a/b^{FL/FL} mice, in the bulge and germ at +10 and +20 days post-tamoxifen is presented. STAT5a/b expression continued to decrease from +10 to +20 days likely because cells with reduced STAT5a/b began to proliferate and predominated the HFSC compartment.

In FIG. 2N, results of immunofluorescence studies for pSTAT5 in WT and K5-CreERT₂::STAT5a/b^{FL/FL} mice 20 days after Tamoxifen induction are presented.

Using this verified mouse model, OSMRβ or STAT5 were conditionally ablated using the K5-CreERT₂ driver during catagen, just before early telogen (P35-P38). Anagen quantification was performed using threshold analysis of the dorsal skin color in ImageJ, because darkening of the skin due to melanogenesis is coupled closely to anagen progression. Mice were closely shaved one day before acquisition of pictures to ensure a close view of the epidermal color. Results in the figures are representative of more than 5 litters for each gene.

STAT5 ablation during catagen prevented HFSC from reaching full quiescence. In the conditional knock-out mice, the second telogen, which typically lasts 40-60 days in the C57BL/6 mouse, was drastically shortened to less than 2 weeks. Interestingly, in the K5-CreERT₂::STAT5a/b^{FL/FL} mice, despite close shaving, their skin remained thick and dark, as if it were still in anagen. (FIG. 2E).

Using the pigmentation of shaved dorsal skin as a surrogate for anagen, K5-CreERT₂::OSMRβ^{FL/FL} and K5-CreERT₂::STAT5a/b^{FL/FL} mice appeared not to enter full telogen as did their wild-type littermates. (FIG. 2F). This was confirmed with H&E immunohistochemistry (IHC) at P60, when their control wild-type (WT) littermates had small telogen HFs that have retracted entirely within the dermis, but mice lacking OSMR or STAT5 in their epidermal compartments were well into their next anagen phase. (FIG. 2O; scale bar 100 μm).

### Example 4: Genetic Ablation of OSMRβ and STAT5 During Telogen in the Epidermis Promotes HFSC Proliferation and Anagen Initiation

Using genetic methods to recapitulate the pharmacological effect of JAK-inhibition in mouse telogen skin, tamoxifen induction was carried out during mid telogen (P56-P60) over a period of 4 days to conditionally knock-out either OSMRβ (K5^{OSMR}) or STAT5 (K5^{STAT5}) in the epidermis and hair follicle. Results are presented in FIGS. 2G and 2H, in which anagen progression was quantified as described in Example 6.3. Results are representative of more than 4 litters for each gene. Data are mean±SEM. *p<0.05, **p<0.01, ***p<0.001, ****p<0.0001, Student's unpaired t-test.

Both K5-CreERT2::OSMRβFL/FL and K5-CreERT2::STAT5a/bFL/FL mice entered anagen significantly earlier than their wild-type or heterozygous littermates, (FIG. 2G), approximately 3 weeks after tamoxifen induction. Quantification of anagen initiation was carried out by the darkening of the dorsal skin of C57BL/6 mice, which reflects activation of melanocyte stem cells, an event that is closely coupled to anagen induction (FIG. 2H) (Muller-Rover, S., et al., A comprehensive guide for the accurate classification of murine hair follicles in distinct hair cycle stages. J Invest Dermatol, 2001. 117(1): p. 3-15). Genetic ablation of OSMRβ or STAT5 in the epidermis and hair follicle after P60 did not result in significant anagen induction.

The cellular dynamics that result from genetic ablation of JAK-STAT5 signaling were studied next. K5-CreERT2::STAT5FL/FL mice and their control littermates were induced with tamoxifen during mid-telogen. Anagen initiation in the STAT5FL/FL mice occurred around 3 weeks after tamoxifen induction, so this perios was bisected to observe the early responses of the HFSCs with STAT5 ablation. Cellular proliferation was studied at 10 and 20 days post-tamoxifen with EdU incorporation (4 mg/25 g adult mouse) into dividing cells 24 hours before the mouse was sacrificed. At 10 days after Cre-recombinase induction, cellular proliferation was observed in the HG, while the bulge remained quiescent. After 20 days, the proliferation in the HG was even more prominent, and EdU incorporation was also seen in the lower bulge. (FIG. 2P; scale bar 100 μm).

Flow cytometric (FACS) quantification of stem cell populations was conducted and showed that the P-cadherin+HG cells significantly doubled (from approximately 0.3% of total epidermal cells to 0.6%) at +10 days post-tamoxifen. (FIG. 2Q). Furthermore, comparing the relative increase in cell numbers between the bulge and HG after STAT5 ablation, the HG proliferated up to 8-fold, while the cell numbers of the bulge stayed relatively constant. (FIG. 2R). This initial proliferation in the HG was consistent with previous reports of the pattern of HFSC activation during spontaneous anagen (Greco, V., et al., A two-step mechanism for stem cell activation during hair regeneration. Cell Stem Cell, 2009. 4(2): p. 155-69), as well as the pattern observed with the administration of topical JAK-inhibitors (Harel, 2015). These genetic data are in agreement with the pharmacological inhibition demonstrated in the earlier Examples, and further show that OSMRβ-JAK-STAT5 signaling is necessary for maintaining HFSC quiescence during murine telogen, and that OSMRβ-JAK-STAT5 maintains this quiescence during the refractory period of telogen (early-to-mid telogen).

### Example 5: Dermal OSM is Produced by Trichophages During Telogen

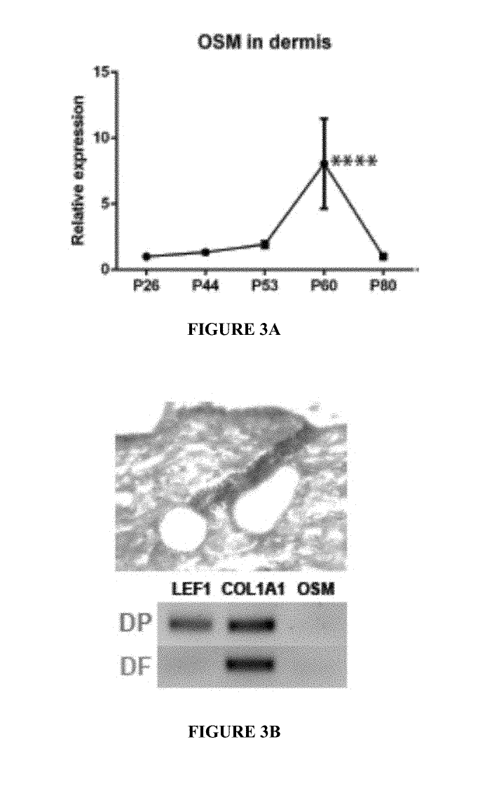

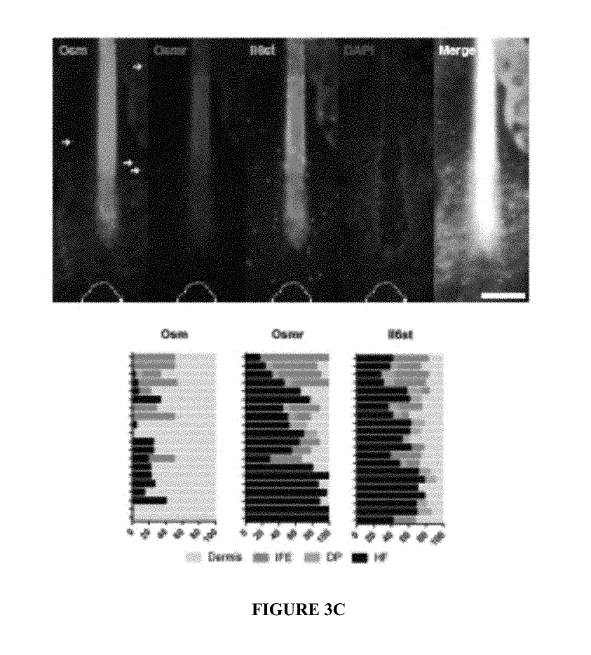

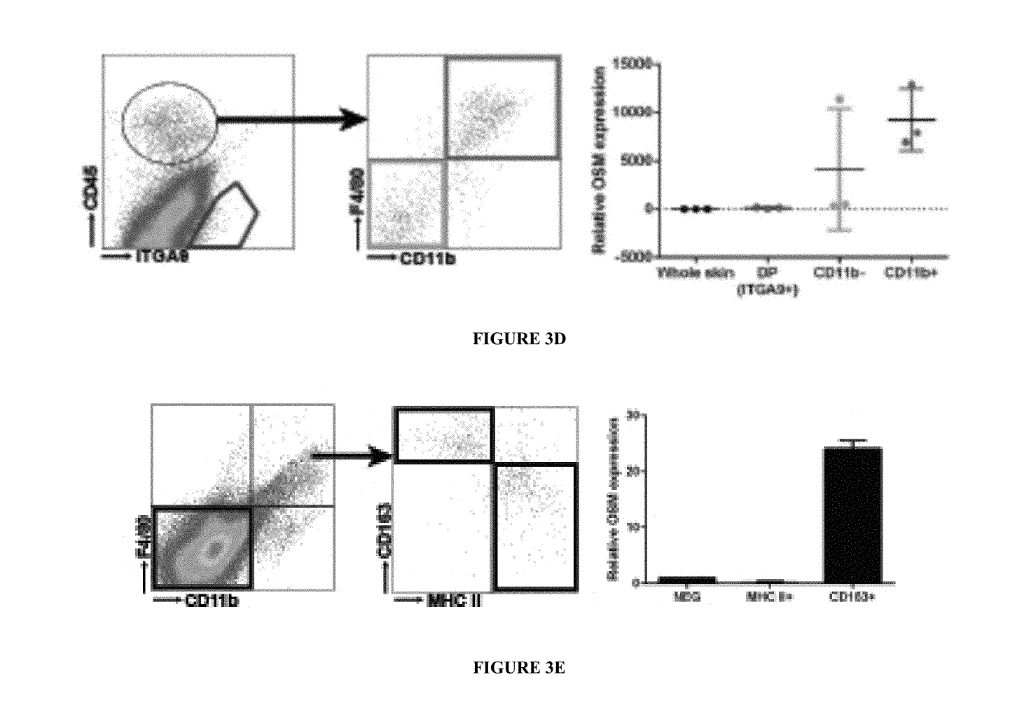

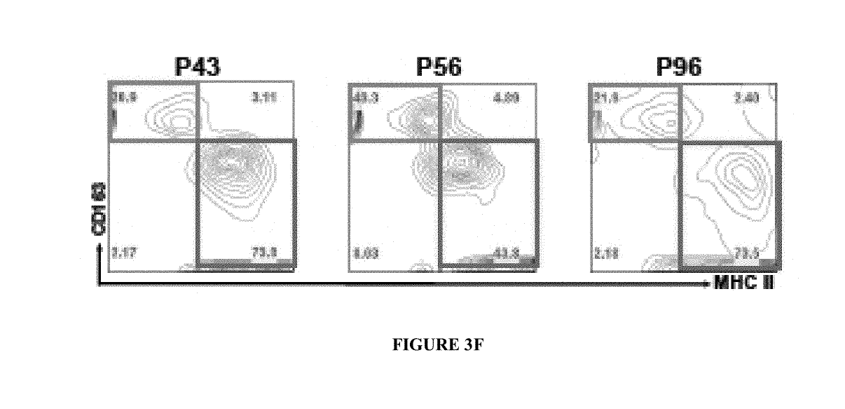

Data in FIG. 3 are mean±SEM. *p<0.05, **p<0.01, ***p<0.001, ****p<0.0001, Brown-Forsyth test of P26 vs all other time points.

FIG. 2A shows that OSM is contained within the dermal fraction (containing the DP), but the source of OSM was unknown. Using qRT-PCR on dermal fractions collected across the murine hair cycle, Osm transcripts were shown to peak in mid telogen (around P60) (FIG. 3A), which mirrors the expression of pSTAT5 seen in FIG. 2C, consistent with its function in maintaining HFSC quiescence.

Previous reports suggested that the human dermal papilla (DP) may be the source for IL-6 (Yu, M., et al., Interleukin-6 cytokine family member oncostatin M is a hair-follicle-expressed factor with hair growth inhibitory properties. Exp Dermatol, 2008. 17(1): p. 12-9), so laser-capture microdissection (LCM) was used to isolate the DP to locate Osm transcripts. Perifollicular dermal tissue, primarily containing fibroblasts (DF) but not DP, was used as a control. However, no Osm mRNA was detected in the DP (dotted green line) or DF (dotted blue line) samples with this method, even though DP-specific Lef1 mRNA was found in the DP samples and Col1a1 mRNA was found enriched in both populations, as expected. (FIG. 3B).

RNAscope multiplex in situ hybridization for Osm, Osmr and Il6st (gene for gp130) was next used to localize the source of OSM. In telogen skin, the transcripts for OSMR and its co-receptor (gp130) were found predominantly within the hair follicle, consistent with previous findings. Given that OSM could not be detected in the perifollicular dermal tissue by LCM, it was reasoned that OSM must be produced by scattered, possibly migratory cells, of the immune system. Using Hair-GEL.net, OSM appears to be expressed in the “Neg” population of skin cells, which includes immune cells, smooth muscle cells and endothelial cells of the dermis (Sennett, R., et al., An Integrated Transcriptome Atlas of Embryonic Hair Follicle Progenitors, Their Niche, and the Developing Skin. Dev Cell, 2015. 34(5): p. 577-91; Rezza, A., et al., Signaling Networks among Stem Cell Precursors, Transit-Amplifying Progenitors, and their Niche in Developing Hair Follicles. Cell Rep, 2016. 14(12): p. 3001-18). In agreement with these reports, Osm transcripts were found scattered in the dermal tissue adjacent to the hair follicle, and not in the DP using multiplex fluorescence in-situ hybridization (RNAscope). Osm mRNA was detected most frequently in perifollicular dermal regions (white arrows). Osmr and Il6st mRNA was generally found in the hair follicle keratinocytes. Quantification was performed over 21 imaged follicles from 2 mice in early-mid telogen. (FIG. 3C).

Tissue macrophages were recently reported to have a role in maintaining the second murine telogen (Castellana, D., R. Paus, and M. Perez-Moreno, Macrophages contribute to the cyclic activation of adult hair follicle stem cells. PLoS Biol, 2014. 12(12): p. e1002002), and were, therefore, investigated as the source of OSM. Using flow-cytometry, the telogen dermis was sorted for macrophages (CD45+ F4/80+ CD11b+). Using qRT-PCR, the non-macrophage immune cells (CD45+ F4/80− CD11b−) and presumptive DP (CD45− ITGA9+, identified using the online resource Hair-GEL.net (Sennett, 2015 and Rezza, 2016) were mostly negative for OSM. Macrophages (CD45+ F4/80+ CD11b+) were found by qRT-PCR to be the main source of dermal OSM. DPs and CD11b− immune cells were generally negative for OSM. (FIG. 3D).

Using CD163 and MHC II as markers for the anti-inflammatory (“M2-like”) and inflammatory (“M1-like”) macrophages respectively, the OSM was found by flow cytometry analysis to be produced by the predominantly anti-inflammatory “M2-like” subset during telogen. (FIG. 3E). Using these same markers across telogen, CD163⁺ “M2-like”) macrophages increased in proportions from early-to-mid telogen (green boxes) and predominated in mid-telogen, coinciding with the period of increased OSM expression, while pro-inflammatory “M1-like” macrophages became less frequent during the same period (pink boxes). (FIG. 3F). Using CD206 as an “M2-like” marker produced identical results.

The “M2-like” macrophages were further characterized herein and identified as trichophages.

### Example 6: Single-Cell RNA-Sequencing of Dermal CD45+ Immune Cells

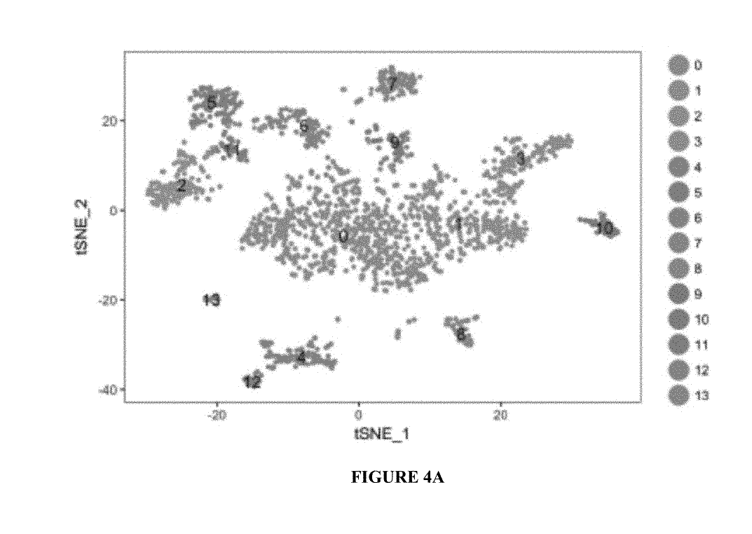

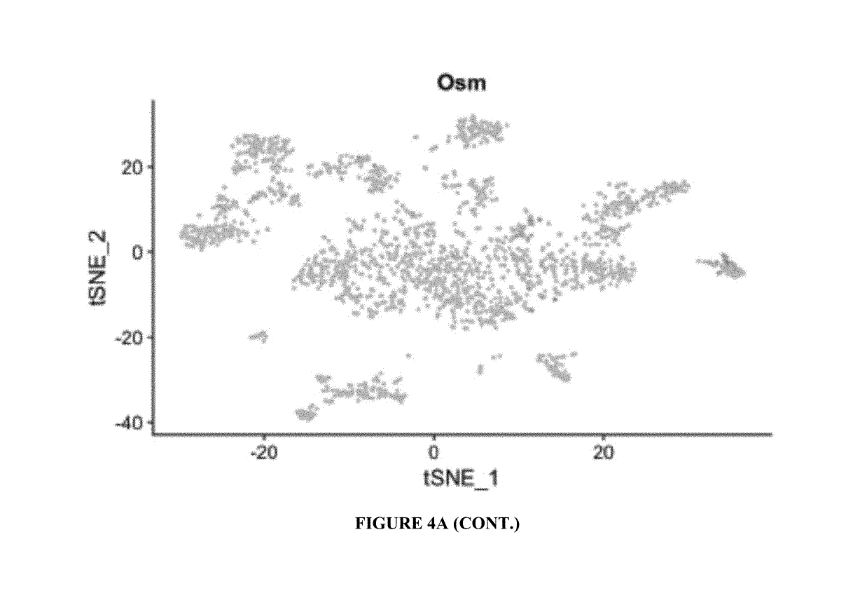

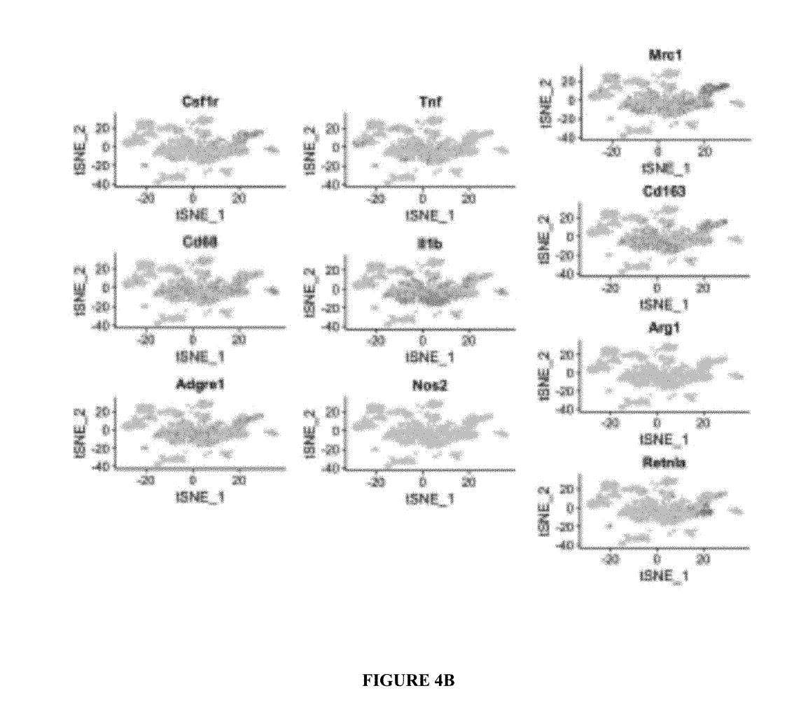

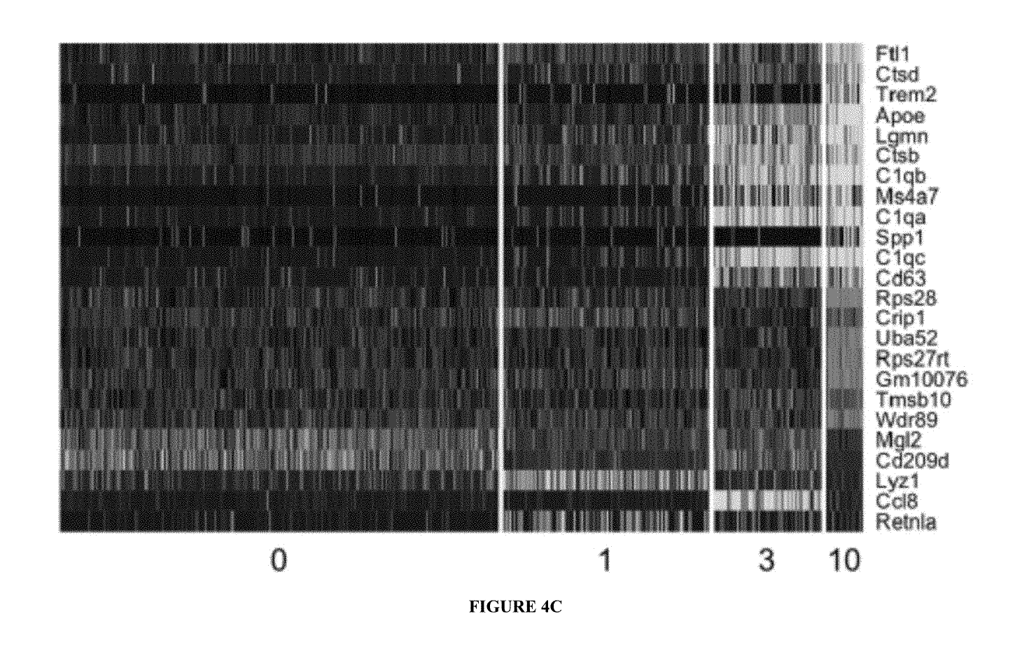

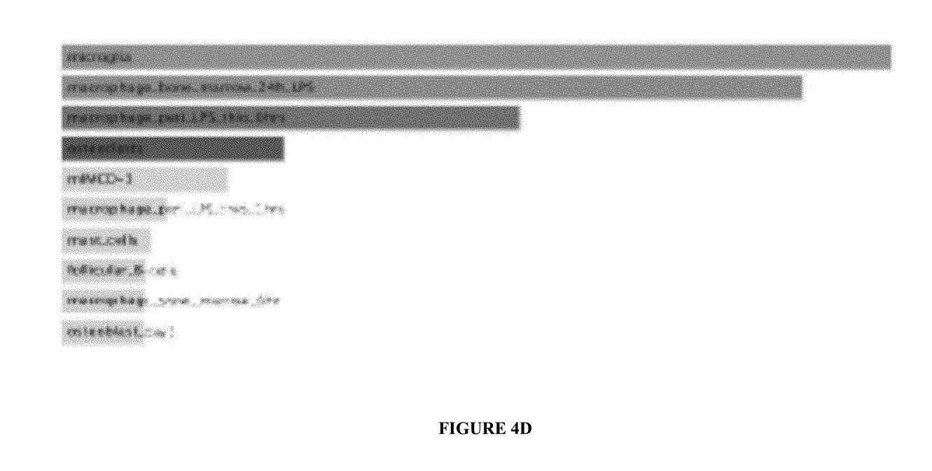

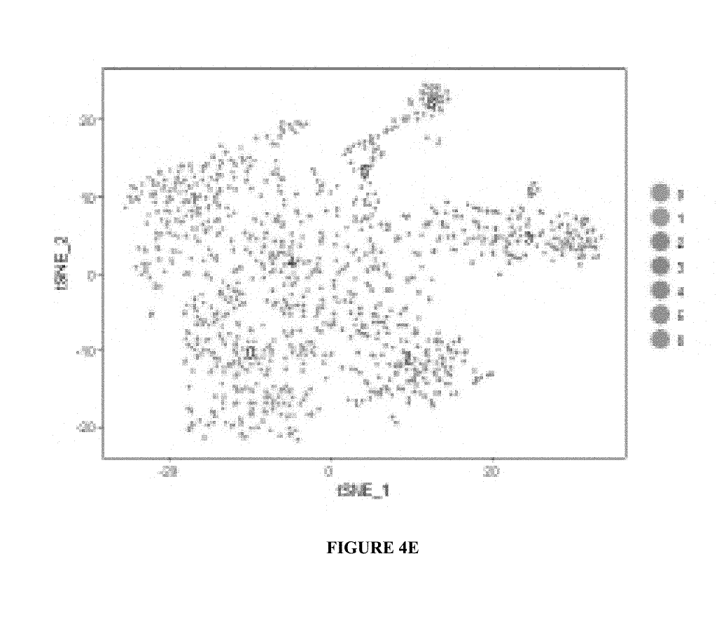

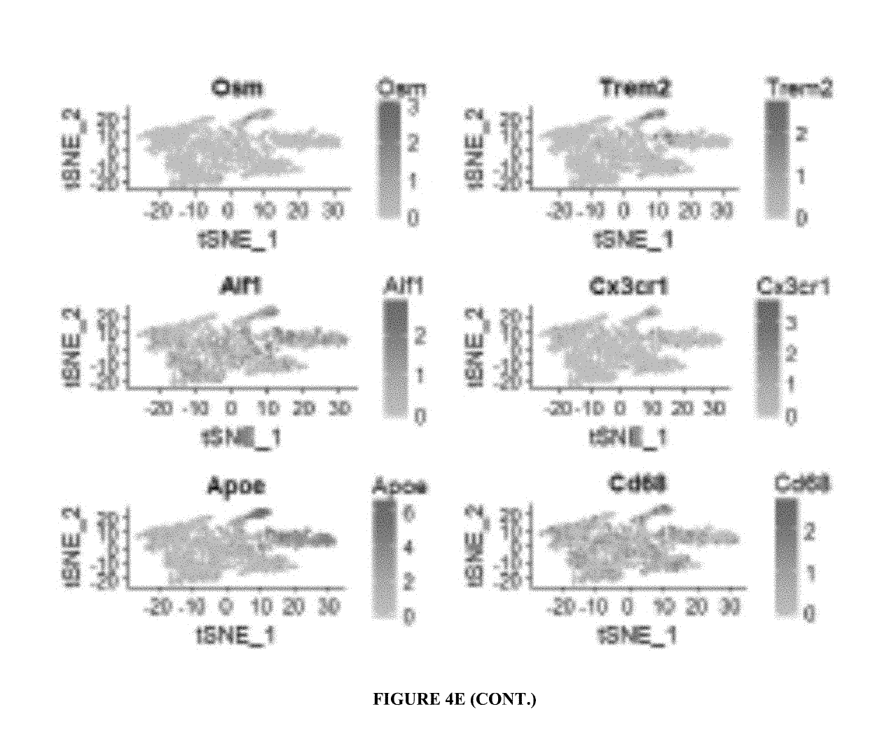

In order to characterize the subset of macrophages that produce OSM in the dermis, single-cell RNA sequencing was performed on the dermal CD45+ immune cells during early (P45), mid (P50, P63) and late (P80) telogen. This provided an unbiased survey of the immune cells across telogen. Analysis of 2145 CD45+ immune cells during early telogen (P45) isolated using flow cytometry and analyzed using the 10× Genomics platform (FIG. 4B) revealed distinct clusters corresponding to T cells (CD3e+) (clusters 2, 5, 6, and 11) and macrophages (CSF1R+, CD68+ and Adgre1/F4/80+) (clusters 0, 1, 3, and 6), which were the main components of the immune infiltrate in unperturbed mouse telogen skin. Macrophages during early telogen clustered into 4 distinct clusters: 0, 1, 3 and 10. M1 markers (TNF, IL-1β, NOS2/iNOS) and M2 markers (Mrc1/CD206, CD163, Arg1, Retn1a) identified clusters 0 and 1 as “M1-like” and cluster 3 as “M2-like”. Cluster 10 did not appear to have a distinct M1 or M2 polarization. OSM itself was significantly upregulated in a distinct cluster (cluster 10). (FIG. 4A). Gene-set enrichment analysis of the macrophage clusters using a heat map showed that OSM-producing macrophages in Cluster 10 were closely related to Cluster 3 macrophages, which were likely “M2-like” macrophages (trichophages) due to the expression of M2 genes like CD206 (MRC1) and CD163. (FIG. 4C).

### Example 7: OSM-Producing Macrophages (Trichophages) have a Distinct Gene-Expression Profile

Using the online resource Enrichr (<<http://amp.pharm.mssm.edu/Enrichr/>>, accessed October, 2017) to analyze the distinguished genes of the OSM-producing subset (including Apoe, TREM2, Ctsd, Ciqa, Clqb, Clqc), demonstrated that this set of genes were associated with microglia, which are the tissue-resident macrophages of the central nervous system. (FIG. 4D). These markers were predominant in the “M2-like” macrophages during early telogen, and TREM2 in particular was specific to the OSM-producing macrophages (trichophages). In particular a TSNE-plot for just macrophages at P45 showed 6 distinct clusters. OSM-producing macrophages were represented by Cluster 6 in this plot. Microglial markers Aif-1/Iba-1, Cx3cr1, Tmem119, ApoE and TREM2 are co-expressed in P45 macrophages, with TREM2 being the most specific to this distinct population. (FIG. 4E).

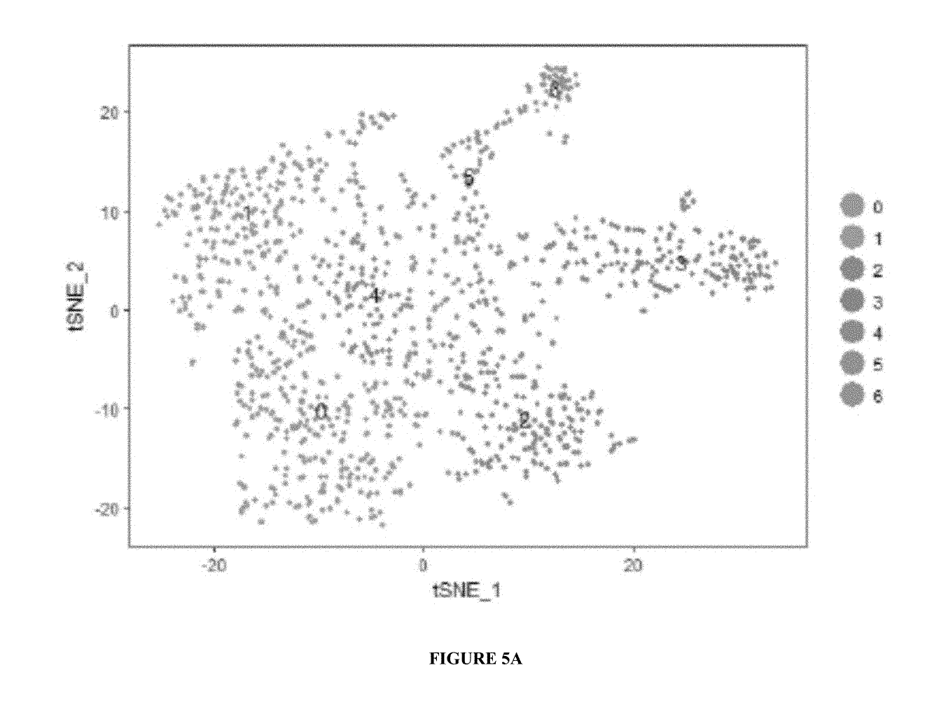

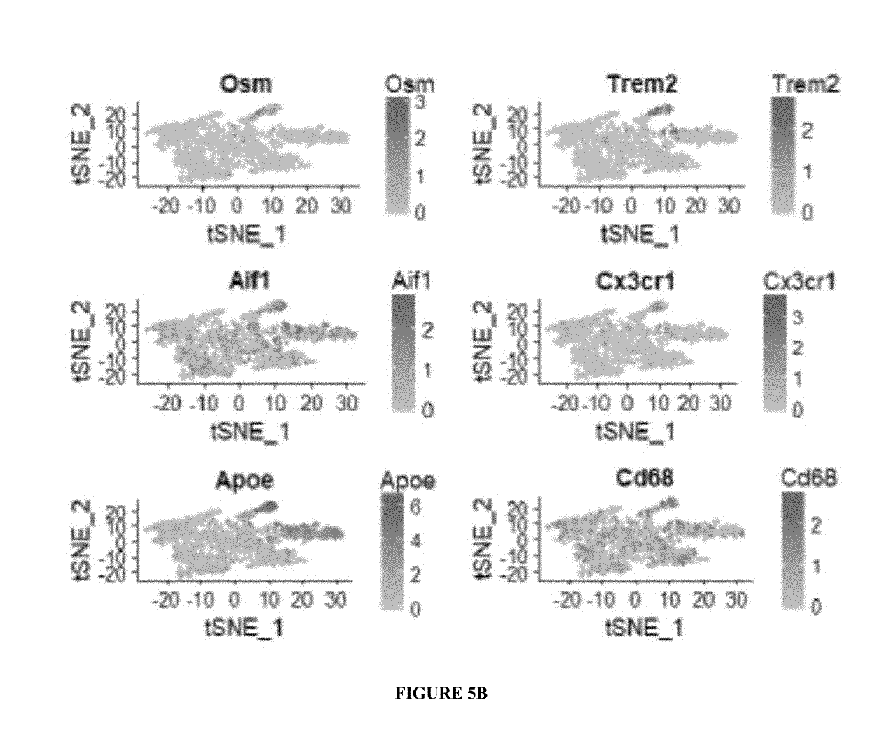

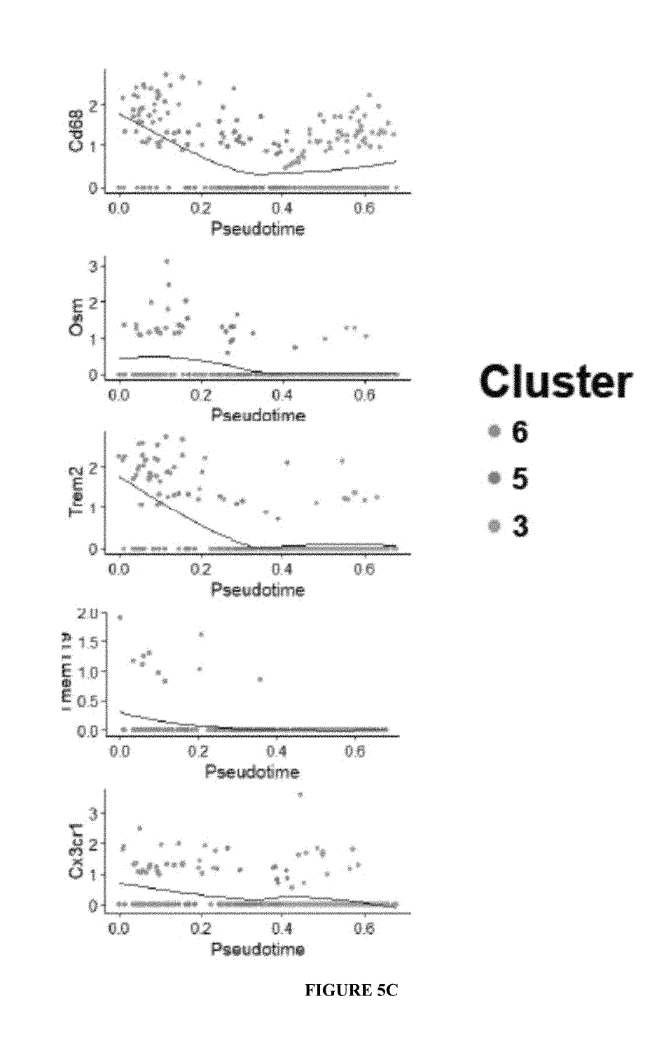

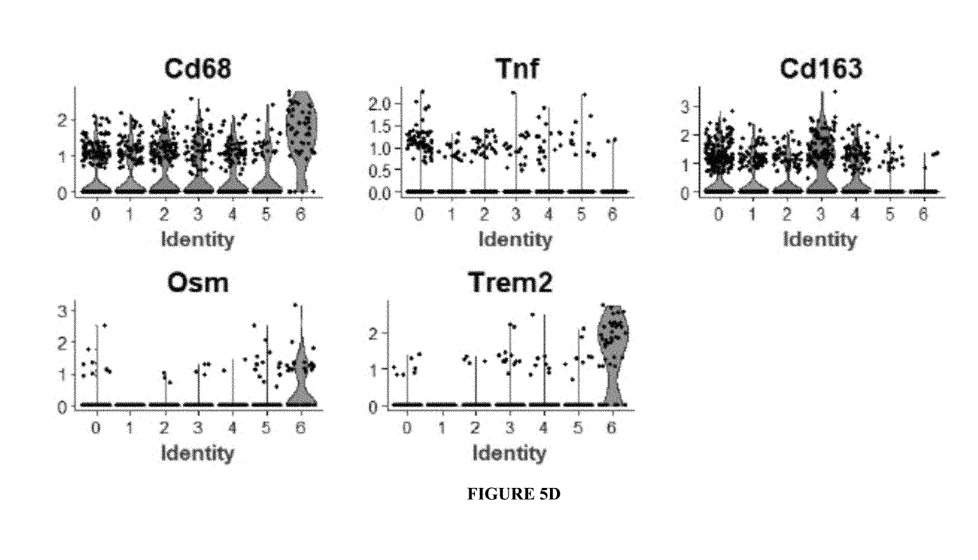

Comparing the scRNA-seq data across three further timepoints in second telogen (P50, P63, P80), the macrophage population was observed to decrease by late telogen, and the specific OSM cluster became less distinct during mid-to-late telogen. (FIG. 5A).

scRNA-seq was also performed on dermal immune cells from a mouse that underwent depilation during mid-telogen (cells were collected 5 days post-depilation, before any noticeable increase in pigmentation). Consistent with OSM-producing macrophages (trichophages) being associated with HFSC quiescence, depilated dorsal skin were found to be devoid of this subset of macrophages. (FIG. 5B).

### Example 8: OSM-Producing Macrophages (Trichophages) are Closely Associated with HFSC

Immunofluorescence studies were carried out with the microglial markers identified in trichophages. Aif-1, which were expressed in most macrophages at P45, was found to co-localize with CD11b and OSM, in immune cells the surrounded the hair follicle at P45 (FIG. 5C). TREM2, which was even more specific for the OSM-producing macrophage subset (trichophages), was also found to co-localize with F4/80 and OSM in close apposition to telogen HFSC. (FIG. 5D). These data combined strongly suggest that OSM is produced by a distinct subset tissue macrophage that is closely associated with HFs, which may have similarities to microglia. This distinct macrophage is identified herein as a trichophage.

### Example 9: Trichophage Inhibition During Telogen Leads to Anagen Initiation

Three independent methods of macrophage inhibition during telogen were used to initiate anagen in C57BL/6 mice.

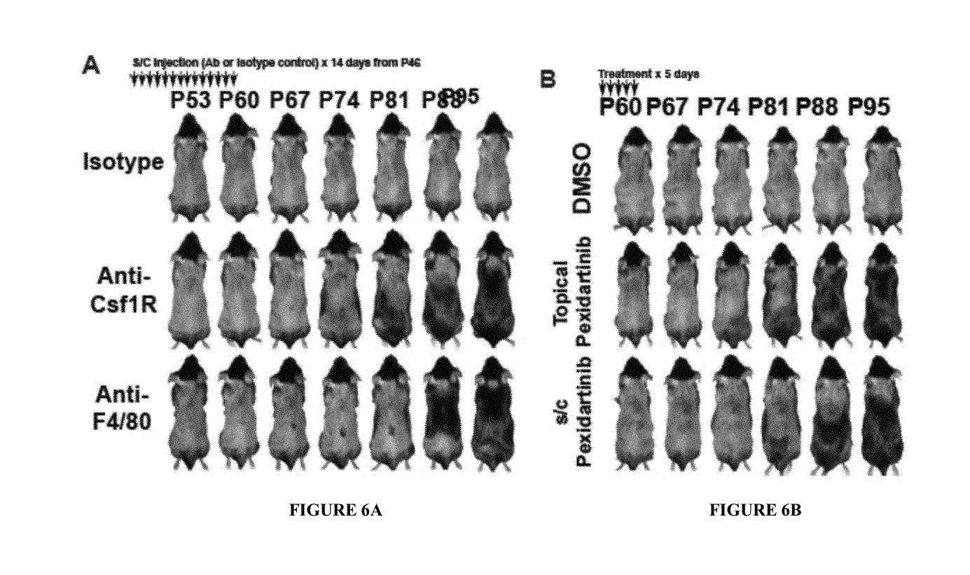

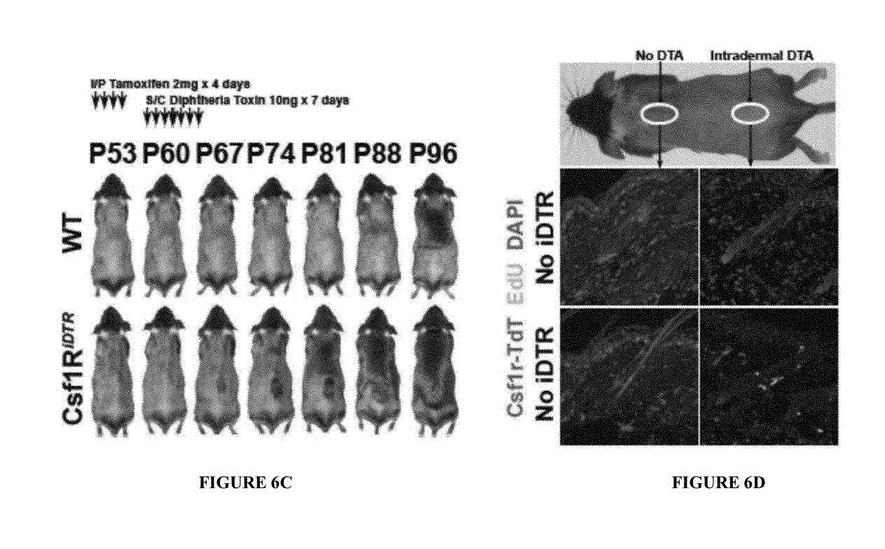

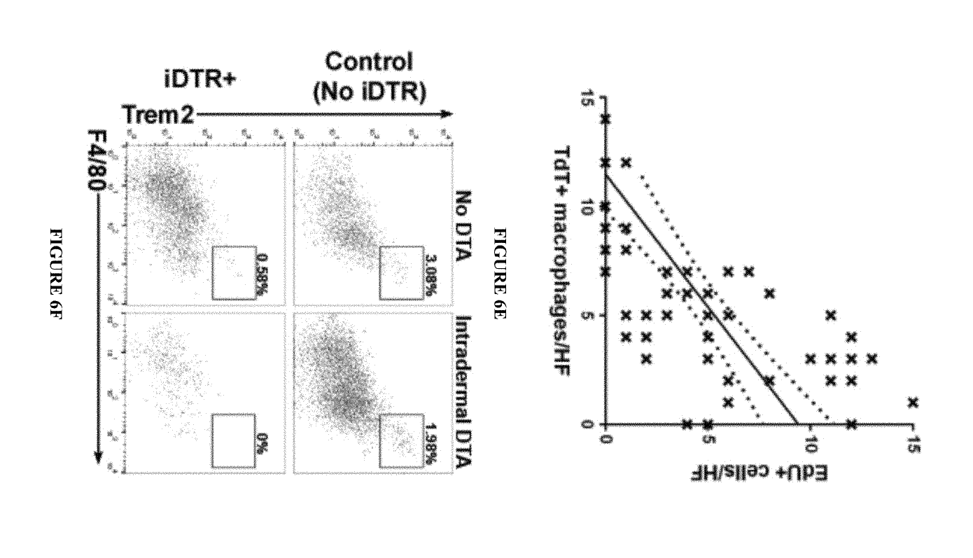

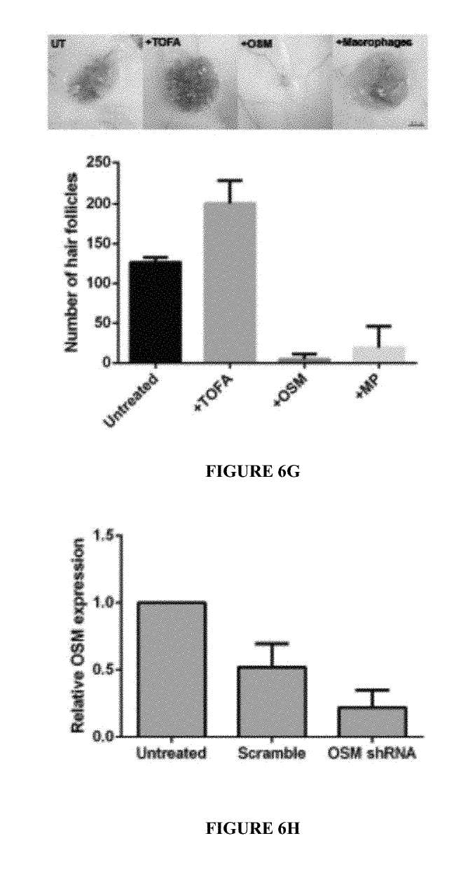

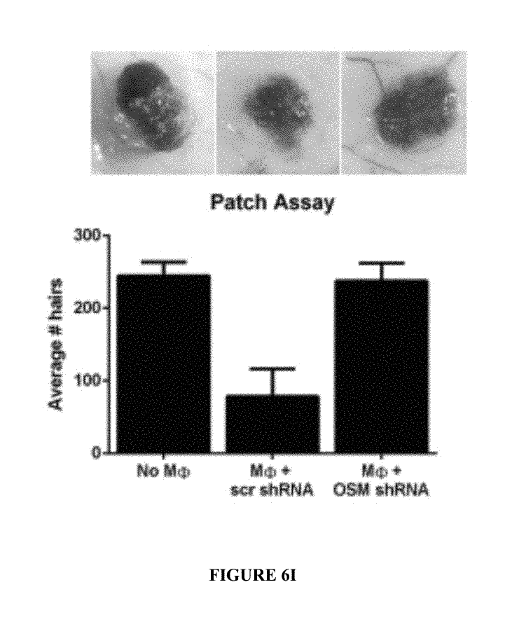

Neutralizing antibodies to CSF1R (AFS98) and F4/80/EMR1 (CI:A3-1) have been shown to preferentially deplete tissue resident populations of macrophages (MacDonald, K. P., et al., An antibody against the colony-stimulating factor 1 receptor depletes the resident subset of monocytes and tissue-and tumor-associated macrophages but does not inhibit inflammation. Blood, 2010. 116(19): p. 3955-63; Segawa, M., et al., Suppression of macrophage functions impairs skeletal muscle regeneration with severe fibrosis. Exp Cell Res, 2008. 314(17): p. 3232-44). Neutralizing antibodies to CSF1R and F4/80 injected intradermally into the middle of the dorsal skin of C57BL/6 mice for 14 days (FIG. 6A) and topical and subcutaneous Pexidartinib (PLX3397, CSF1R tyrosine kinase inhibitor) administration for 5 days (FIG. 6B) were both successful at initiating anagen when administered during mid-telogen.

A CSF1R-CreER mouse was bred with Rosa26-iDTR mice, producing offspring (CSF1R-CreER::R26-iDTR mice) that express the diphtheria toxin receptor on CSF1R+ cells upon tamoxifen induction. Tamoxifen was administered for 4 days in mid-telogen (from P53-P56), followed by 7 days of 10 ng intradermal diphtheria toxin (DTA) injections starting at P60. This treatment led to ablation of dermal macrophages, and resulted in local anagen initiation that preceded their the wild-type littermates that received the same treatments. (FIG. 6D).

Introduction of the R26-TdTomato reporter (CSF1R-CreERT::R26-TdTomato::R26-iDTR mice) allowed macrophage ablation in these mice to be demonstrated. 10 ng intradermal DTA was administered and dermis from area of injection and a remote area on the dorsal skin were collected and examined with IF and FACS. Intradermal DTA was associated with a drastic reduction in perifollicular TdT+ macrophages, and this was associated with increased EdU incorporation of HFSCs when quantification was carried over 50 HFs across 2 mice. (FIG. 6D; Dotted lines represent 95% CI). Ablation of HF-associated macrophages was strongly correlated with increased HFSC proliferation (FIG. 6E). Flow cytometry of the dermis in these areas also showed ablation of the F4/80+/TREM2+ subset of macrophages (FIG. 6F), which correspond to the OSM-producing macrophage subset (trichophages).

T regulatory cells, which have recently been shown to play a role in depilation-induced anagen (Ali, N., et al., Regulatory T Cells in Skin Facilitate Epithelial Stem Cell Differentiation. Cell, 2017. 169(6): p. 1119-1129 ell), were depleted using anti-CD25 neutralizing monoclonal antibody (PC61). These mice as well as mice treated with the small molecule CSF1R tyrosine kinase inhibitor Pexidartinib to target macrophages initiated anagen in the same manner as wild-type mice treated with a JAK-inhibitor. (FIG. 6I).

These data, are consistent with and build upon previously published findings (Castellana, D., R. Paus, and M. Perez-Moreno, Macrophages contribute to the cyclic activation of adult hair follicle stem cells. PLoS Biol, 2014. 12(12): p. e1002002) and strongly establish that macrophages, and in particular the newly identified trichophages discussed herein, are necessary for the maintenance of an inhibitory environment on the HFSCs during telogen, likely by producing OSM.

### Example 10: Trichophage OSM Inhibits Hair Regeneration

To examine the interactions between trichophages and HFSC in more detail, hair reconstitution assays were used to show that trichophages had an inhibitory effect on hair regeneration. Neonatal keratinocytes and dermal cells were injected intradermally in a 1:2 ratio in the patch assay, which recapitulates the epithelial-mesenchymal interactions required for HF regeneration. While tofacitinib enhanced the hair follicles generated in this assay, consistent with previous findings (Harel, 2015), addition of OSM was sufficient to nearly completely inhibit hair reconstitution. F4/80+ CD11b+ trichophages sorted from the P60 adult telogen dermis were recombined with the neonatal cells, and these were also found to inhibit hair reconstitution. (FIG. 6G).

In order to determine whether OSM in the trichophages were responsible for their inhibitory effects, murine peritoneal macrophages obtained with gavage were cultured in the presence of M-CSF to skew them to a tissue-resident, anti-inflammatory phenotype, which would include trichophages using the methods of Weisser, S. B., et al., Generation and characterization of murine alternatively activated macrophages. Methods Mol Biol, 2013. 946: p. 225-39. Plasmids containing shRNA (scrambled or OSM-specific) were transfected into the macrophages to knock-down OSM expression (when OSM-specific shRNA was used) and PSM production was analyzed by qRT-PCR. (FIG. 6H). While macrophages with scrambled shRNA were still able to inhibit hair reconstitution, knock-down of OSM in macrophages attenuated the inhibitory properties of the macrophages, and restored the original hair reconstitution in a patch assay. (FIG. 6I). These data show that OSM produced by trichophages is the cytokine that inhibits proliferation and activation of HFSCs.

### Example 11: Trichophages and OSM are Associated with Androgenetic Alopecia

Following the reasoning that miniaturized hairs in androgenetic alopecia (AGA) may be a pathological form of arrested telogen, RNA-seq was performed on balding versus non-balding scalp for AGA patients (3 samples in each group). The top 11 differentially expressed and elevated genes are shown in Table 2.

TABLE 2
Differentially expressed genes in balding and non-balding scalps

| | Fold Change balding | |
|---|---|---|
| Gene | v. non-balding | p-value |
| FCGR1A | 49.50 | 4.604E−02 |
| CSF3 | 20.29 | 9.255E−03 |
| OSM | 18.94 | 1.456E−02 |
| PROK2 | 15.43 | 4.343E−02 |
| EMR1 | 13.40 | 4.998E−03 |
| ADAMTS4 | 13.06 | 3.494E−02 |
| PAX8 | 12.03 | 8.106E−05 |
| PLAUR | 9.10 | 3.903E−02 |
| CD300C | 8.63 | 4.534E−02 |
| P13 | 8.60 | 2.994E−02 |
| CCR1 | 8.40 | 4.733E−03 |

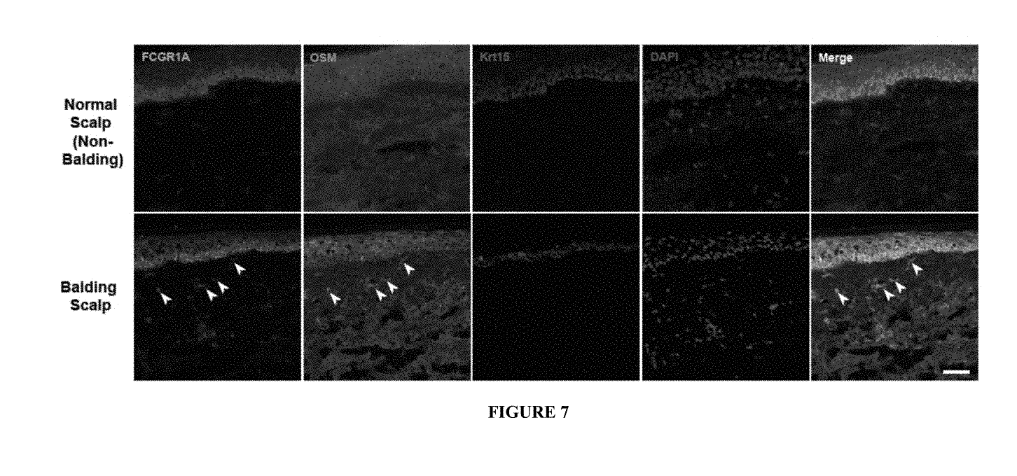

Interestingly, the six top enriched genes in balding scalp were related to macrophages and monocytes (FCGR1A, CSF3, EMR1, ADAMTS4, CD300C, CCR1). Notably, OSM itself was markedly increased 19-fold in AGA balding scalp and was the third most upregulated gene. (FIG. 7). Immunofluorescence studies confirmed an influx of CD11b+ and FCGR1A+ positive cells in the dermis of balding AGA patients, and that these cells contained with OSM, which co-localized with CD11b+ dermal cells. (FIG. 7; bottom row; scale bar 50 μm). In normal and non-balding scalp, CD11b+ OSM+ cells tended to be restricted to dermal blood vessels. (FIG. 7; top row; scale bar 50 μm). These data suggest the trichophages, and the OSM that they produce, are physiologically relevant in the suppression of human HFSC activation, and may play a pathogenic role in AGA.

### Example 12: Clinical Response to Treatment of Androgenetic Alopecia (AGA) with Inhibitors of Oncostatin M (OSM), Colony Stimulating Factor 1 Receptor (CSF1R), Interleukin 34 (IL-34), and/or Trichophages

Methods for assessing the efficacy of a therapy for promoting hair growth, inducing hair growth, maintaining the rate of hair growth, increasing the rate of hair growth, decreasing the rate of hair loss, preventing the onset or progression of a hair loss disorder, maintaining remission in a subject having a hair loss disorder, improving remission in a subject having a hair loss disorder, preventing hair loss, or the like in a mammalian subject by clinically evaluating hair growth during a therapy are not therapy specific and are well known in the art. Scales or tools that are well known in the art include quality of life scales such as the Dermatology Life Quality Index (DLQI) score and additional scales/tools such as e.g., for the evaluation of AGA, the Norwood-Hamilton scale in males and the Sinclair Scale in females, and in AA and its variants (and other hair-loss disorders), tools such as the Severity of Alopecia Tool (SALT) score or Alopecia Density and Extent Score (ALODEX) score. Additional scales/tools for clinical evaluation include the Alopecia Scalp Appearance Assessment (ASAA) Patient-reported outcome [PRO] Scale, the Alopecia Scalp Appearance Assessment (ASAA) Clinician-reported outcome [ClinRO] Scale, a Physicians Global Impression of Severity (PhGIS) scale, a Subject Global Impression of Severity (SGIS) scale, a subject reported Alopecia Impact Assessment (AIA) scale, a subject Global Impression of Treatment Satisfaction (SGITS) scale, a Subject Global Satisfaction with Hair Quality (SGSHQ) scale, a Global Impression of Change (Clinician and Subject) tool, and other assessments that may include hair quality assessments such as hair thickness.

It is anticipated that patients treated locally or systemically with a therapeutically effective amount of an inhibitor of oncostatin (e.g. oncostatin M (OSM)), inhibitor of colony stimulating factor 1 receptor (CSF1R), interleukin 34 (IL-34) inhibitor, and/or trichophage inhibitor alone, in combination, as disclosed in the above embodiments will show clinical improvement as judged by the patient and/or their physician and/or will show improvement in their hair loss condition as measured by one or more of the scales or tools described above.

Various publications, patents and patent application are cited herein, the contents of which are hereby incorporated by reference in their entireties.

1. A method of treating a hair loss disorder in a mammalian subject, the method comprising administering to the subject a therapeutically effective amount of a CSF1R, oncostatin, IL-34, and/or trichophage inhibitor.

2. The method of claim 1, wherein the hair loss disorder is selected from androgenetic alopecia (AGA), non-scarring alopecia, scarring alopecia, male and female pattern AGA, alopecia areata (AA), alopecia totalis (AT), alopecia universalis (AU), eyebrow alopecia, eyelash alopecia, intranasal hair alopecia, ophiasis pattern alopecia areata, sisaihpo pattern alopecia areata, male pattern hair loss, female pattern hair loss, anagen effluvium, telogen effluvium, hypotrichosis, hereditary hypotrichosis simplex, frontal fibrosing alopecia, cicatricial alopecia, lichen planopilaris, folliculitis decalvans, tufted folliculitis, dissecting cellulitis of the scalp, ring alopecia, chemotherapy induced alopecia, superficial or deep infections of the scalp, or tinea capitis.

3. The method of claim 1, wherein the inhibitor is an antisense RNA, an siRNA, an shRNA, a microRNA, or a variant or modification thereof that specifically inhibits expression of the gene that encodes CSF1R; or a small molecule.

4. The method of claim 1, wherein the inhibitor is selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

5. The method of claim 1, wherein the inhibitor is an CSF1R antibody, a CSF1 antibody, a IL-34 antibody selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), and a combination thereof.

6. The method of claim 1, wherein the subject is a human.

7. The method of claim 1, wherein the inhibitor is administered locally, systemically, topically, orally, intradermally, intramuscularly, intraperitoneally, intravenously, subcutaneously, by intra-pulmonary administration, or by injection.

8. The method of claim 1, wherein administration is to an alopecic area of the body.

9. The method of claim 1, wherein administration is to a head, a scalp, a face, an eyebrow area, nasal hair area, or an eyelash area of the subject.

10. The method of claim 1, wherein an expression level of one or more hair growth biomarkers, CSF1R, and/or one or more trichophage biomarkers are changed after administering said inhibitor.

11. The method of claim 10, wherein the one or more hair growth biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

12. The method of claim 10, wherein the expression level change of one or more biomarkers are detected by quantitative PCR, RNA sequencing, single-cell RNA sequencing, enzyme linked immunosorbant assay, or a variation thereof.

13. A method of inducing or promoting hair growth in a mammalian subject, the method comprising administering to the subject a therapeutically effective amount of a CSF1R inhibitor, an oncostatin inhibitor, an IL-34 inhibitor, and/or trichophage inhibitor.

14. The method of claim 13, wherein the subject has androgenetic alopecia (AGA), non-scarring alopecia, scarring alopecia, male and female pattern AGA, alopecia areata (AA), alopecia totalis (AT), alopecia universalis (AU), eyebrow alopecia, eyelash alopecia, intranasal hair alopecia, ophiasis pattern alopecia areata, sisaihpo pattern alopecia areata, male pattern hair loss, female pattern hair loss, anagen effluvium, telogen effluvium, hypotrichosis, hereditary hypotrichosis simplex, frontal fibrosing alopecia, cicatricial alopecia, lichen planopilaris, folliculitis decalvans, tufted folliculitis, dissecting cellulitis of the scalp, ring alopecia, chemotherapy induced alopecia, superficial or deep infections of the scalp, or tinea capitis.

15. The method of claim 13, wherein the inhibitor is an antisense RNA, an siRNA, an shRNA, a microRNA, or a variant or modification thereof that specifically inhibits expression of the gene that encodes CSF1R; or a small molecule.

16. The method of claim 13, wherein the inhibitor is selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

17. The method of claim 13, wherein the inhibitor is a CSF1R antibody, a CSF1 antibody, an IL-34 antibody selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), and a combination thereof.

18. The method of claim 13, wherein the subject is a human.

19. The method of claim 13, wherein the inhibitor is administered locally, systemically, topically, orally, intradermally, intramuscularly, intraperitoneally, intravenously, subcutaneously, by intra-pulmonary administration, or by injection.

20. The method of claim 13, wherein administration is to an alopecic area of the body.

21. The method of claim 13, wherein administration is to a head, a scalp, a face, an eyebrow area, nasal hair area, or an eyelash area of the subject.

22. The method of claim 13, wherein an expression level of one or more hair growth biomarkers, CSF1R, and/or one or more trichophage biomarkers are changed after administering said inhibitor.

23. The method of claim 22, wherein the one or more hair growth biomarkers are selected from the group consisting of CD34, Lhx2, NFATc1, Axin2, FoxC1, OSMR, OSM, Jak3, FAS, Irf1, Ifnar1, Nr3c1, Stat5A, Il6st, Ptprc, Ghr, IL10ra, Il2rg, Pdgfra, Spfi1, Socs2, Stat5b, Crp, Il4, Prlr, Insr, IL2ra, Cebpd, Stat3, Jak1, Acvr2a, Sfrp4, Sox5, Cdh2, Fzd5, Wif1, Wnt2, Fzd8, Apc, Sox9, Ilk, Shh, Krt25, Dlx2, Prom1, S100a9, Vegfc, Ptgfr, Pdgfr1, Igfbp4, Gli2, Tyrp1, Syt4, Mlana, Pme1, Dct, Tyr, Sos1, Dbf4, Pax3, PIK3ca, Rps6kb1, Mlph, and Stx17.

24. The method of claim 22, wherein the expression level change of one or more biomarkers are detected by quantitative PCR, RNA sequencing, single-cell RNA sequencing, enzyme linked immunosorbant assay, or a variation thereof.

25. A kit for inducing or promoting hair growth in a mammalian subject, the kit comprising:
(a) a CSF1R, IL-34, oncostatin, and/or trichophage inhibitor; and
(b) a pharmaceutically acceptable carrier.

26. The kit of claim 25, wherein the inhibitor is selected from pexidartinib (PLX3397); 5-[(5-chloro-1H-pyrrolo[2,3-b]pyridin-3-yl)methyl]-N-[[6-(trifluoromethyl)pyridin-3-yl]methyl]pyridin-2-amine), 4-cyano-N-(2-(4,4-dimethylcyclohex-1-en-1-yl)-6-(2,2,6,6-tetramethyl-tetrahydro-2H-pyran-4-yl)pyridin-3-yl)-1H-imidazole-2-carboxamide (JNJ-40346527), PLX5622 (selective CSF1R inhibitor manufactured by Plexxikon, Inc.), 4-cyano-N-(2-(1-cyclohexen-1-yl)-4-(1-((dimethylamino)acetyl)-4-piperidinyl)phenyl)-1H-imidazole-2-carboxamide (JNJ-28312141), 5-(3-methoxy-4-((4-methoxybenzyl)oxy)benzyl)pyrimidine-2,4-diamine (GW2580), PLX7486, DCC-3014 (manufactured by Deciphera Pharmaceuticals), PLX73086 (CSF-1R inhibitor manufactured by Plexxikon, Inc.), ARRY382 (CSF1R inhibitor developed by Array BioPharma), 4-[[2-[[(1R,2R)-2-hydroxycyclohexyl]amino]-1,3-benzothiazol-6-yl]oxy]-N-methylpyridine-2-carboxamide (BLZ945); N-[4-[(6,7-Dimethoxy-4-quinolinyl)oxy]-2-methoxyphenyl]-N′-[1-(2-thiazolyl)ethyl]urea ((KI-20227)- a potent and orally active inhibitor of c-Fms tyrosine kinase (M-CSFR, CSF1R)); SNDX-6352 (an IgG4 humanized monoclonal antibody that binds to the ligand binding domain of the CSF-1 receptor, blocking the binding and consequent activation by both natural ligands (IL-34 and CSF-1)), a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, a tautomer thereof, a mixture of tautomers thereof, or a combination thereof.

27. The kit of claim 25, wherein the inhibitor is a CSF1R antibody, a CSF1 antibody, an IL-34 antibody selected from the group consisting of AFS98, cabiralizumab (such as FPA008 developed by Five Prime/BMS), AMG820, IMCCS4 (LY3022855), emactuzumab (such as RG7155 developed by Genentech/Roche), MCS110 (Novartis), PD-0360324 (Pfizer), and a combination thereof.
